(19) United States
(12) Patent Application Publication
(10) Pub. No.: US 2003/0167485 A1
(43) Pub. Date: Sep. 4, 2003

(54) Novel G protein-coupled receptor encoding gene and diagnostic uses therefor

(75) Inventors: Herbert Herzog, Bondi (AU); Robert Lyndsay Sutherland, Lindfield (AU); Charles R. MacKay, Vaucluse (AU); Susan Henshall, Mosman (AU)

Correspondence Address: ROTHWELL, FIGG, ERNST & MANBECK, P.C., 1425 K STREET, N.W., SUITE 800, WASHINGTON, DC, 20005, US

(73) Assignee: Garvan Institute of Medical Research, Darlinghurst (AU)

(21) Appl. No.: 10/073,054

(22) Filed: Feb. 12, 2002

Related U.S. Application Data
(63) Continuation-in-part of application No. 09/308,696, filed on Jun. 11, 1999, now abandoned.
Application No. 09/308,696 is a 371 of international application No. PCT/AU98/00805, filed on Sep. 24, 1998.

(30) Foreign Application Priority Data
Sep. 24, 1997 (AU) PO9386

(51) Int. Cl. A01K 67/00; C12Q 1/68; G01N 33/574; C07H 21/04; C12N 9/00; C12P 21/02; C12N 5/06

(52) U.S. Cl. 800/10; 435/7.23; 435/320.1; 435/69.1; 435/183; 435/325; 536/23.2; 435/6

(57) ABSTRACT

The present invention provides GPR56 genes and polypeptides encoded therefor. The GPR56 genes of the invention are expressed highly in malignant tissues and the invention also provides diagnostic processes for detecting cancer or malignant tumors in human subjects The diagnostic and prognostic test of the present invention is particularly useful for the early detection of ovarian cancer or metastases thereof and for monitoring the progress of disease, such as, for example, during remission or following surgery or chemotherapy. The GPR56 gene of the invention is also expressed in effector memory T cells, and the invention therefor provides methods of identifying and/or quantifying effector memory T cells, which methods are useful for determining the immune status of a subject.

### RELATED APPLICATION DATA

This application is a continuation-in-part application of U.S. Ser. No. 09/308,696 filed on Jun. 11, 1999, which is a 371 of International Application No. PCT/AU98/00805 filed on Sep. 24, 1998, which claims the benefit of priority under Title 35 U.S.C. 119 from Australian Patent Application No. PO 9386 filed on Sep. 24, 1997. These applications are incorporated herein by reference in their entirety and for all purposes.

### FIELD OF THE INVENTION

This invention relates to novel genes that are expressed highly in malignant tissues and uses therefor in the diagnosis of cancer or malignant tumors in human subjects. More specifically, this invention relates to the use of nucleic acid or antibody probes to specifically detect over-expression of a G protein-coupled receptor gene in ovarian cells, such as, for example, the ovarian surface epithelium, which over-expression is highly associated with the occurrence of ovarian tumors. The diagnostic and prognostic test of the present invention is particularly useful for the early detection of ovarian cancer or metastases thereof, or other cancers, and for monitoring the progress of disease, such as, for example, during remission or following surgery or chemotherapy. The present invention is also directed to methods of therapy wherein GPR56 activity is modulated.

### BACKGROUND OF THE INVENTION

1. General

This specification contains nucleotide and amino acid sequence information prepared using Patentln Version 3.1, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein, which are intended for the purposes of exemplification only. Functionally-equivalent products, compositions and methods are clearly within the scope of the invention, as described herein.

2. Description of the Related Art

The actions of many extracellular signals are mediated by the interaction of a G protein-coupled receptor (hereinafter “GPCR” or “GPR”) with its cognate guanine nucleotide-binding regulatory protein (G protein). G protein-mediated signaling systems have been identified in many divergent organisms, such as mammals and yeast. GPCRs respond to, among other extracellular signals, neurotransmitters, hormones, odorants and light (Watson, S. and Arkinstall, S., The G-protein Linked receptor facts Book, Academic Press, London, 1994).

The movement and biological activities of leukocytes (i.e. neutrophils, monocytes, eosinophils, basophils, lymphocytes, dendritic cells, etc.) in the ontogeny of cancer, including the mobilization of hematopoietic stem cells in chemotherapy or myeloprotection during chemotherapy, is also regulated by the GPCR-mediated activity of chemokines. Similarly, GPCR-mediated chemokine activity regulates the movement and biological activities of leukocytes during chronic inflammation, chronic rejection of transplanted organs or tissue grafts, chronic myelogenous leukemia, and infection by HIV and other pathogens. For reviews, see Baggiolini et al. (1997) Ann. Rev. inmmunol. 15, 675-705; Premack et al., Nature Medicine 2, 1174-1178, 1996; and Yoshie et al., J. Leukocyte Biol. 62, 634-644, 1997). Over 30 different human chemokines have been described to date, which vary in specificity for different leukocyte types, however they typically are produced at sites of tissue injury or stress, where they promote infiltration of leukocytes to facilitate an inflammatory response. Some chemokines act selectively on immune system cells such as subsets of T cells or B lymphocytes or antigen presenting cells, and may thereby promote immune responses to antigens. Some chemokines also have the ability to regulate the growth or migration of hematopoietic progenitor and stem cells that normally differentiate into specific leukocyte types, thereby regulating leukocyte numbers in the blood.

In consideration of the role of GPCRs in regulating chemokine activity, the identification of immunodominant T cell epitopes and enumeration of frequencies of T cell sub-populations is a particularly desirable outcome. Known means for achieving this end include a modified proliferation assay (Plebanski, et al., J. Immunol. Meth. 170,15, 1994), a limiting dilution assay (LDA) employing relatively large peripheral blood mononuclear cell (PBMC) quantities and requiring two rounds of in vitro stimulation to detect a T cell response to whole antigen or peptide (Sharrock et al., Immunol. Today 11, 281-286, 1990), and several formats of flow cytometric methods to detect T cell activation by up-regulation of characteristic markers such as CD69, measurement of lymphokine production (Jung et al., J. Immunol. Meth. 159, 197, 1993), or by trapping secreted lymphokines on the surface of the secreting cell (Manz et al., Proc. Natl. Acad. Sci. USA 92, 1921, 1995). The identification of T cell markers is particularly important to facilitating the identification of immunodominant T cell epitopes and enumeration of frequencies of T cell sub-populations. T cell markers act as surrogate markers for disease activity, indicating the significance of GPR56 in diagnostic, prognostic, and therapeutic applications with respect to hyperproliferative disorders or inflammatory disease.

Several GPCRs have been identified and sequence analysis reveals that they are structurally similar, possessing a number of highly conserved amino acid residues. Accordingly, GPCRs collectively form a large “superfamily” of receptor proteins capable of associating with the plasma membrane such that the N-terminal portion is localized in the extracellular space, the C-terminus is cytoplasmic, and three extramembranous loops are forms. This means that there are seven transmembrane domains (i.e. 7-TM) in the GPCR polypeptide.

Individual GPCR types activate particular signal transduction pathways. At least ten different signal transduction pathways are known to be activated via GPCR polypeptides. For example, the secretin receptor sub-family of GPCR polypeptides are activated by a ligand selected from the group consisting of: secretin, glucagon, calcitonin, glucagon-like peptide 1, parathyroid hormone, parathyroid-related peptide, corticotropin-releasing factor (CRF), growth hormone-releasing hormone (GHRH), gastric inhibitory polypeptide, pituitary adenylate cyclase-activating polypeptide (PACAP), vasoactive intestinal peptide (VIP), and insect diuretic hormone (DHR). Known human chemokine receptors include CCR1, CCR2, CCR3, CCR4, CCR5, CCR6, CCR7, CCR8, CXCR1, CXCR2, CXCR3, and CXCR4.

Cancer is a multi-factorial disease and major cause of morbidity in humans and other animals, and deaths resulting from cancer in humans are increasing and expected to surpass deaths from heart disease in future. Carcinomas of the lung, prostate, breast, colon, pancreas, and ovary are major contributing factors to total cancer death in humans. For example, prostate cancer is the fourth most prevalent cancer and the second leading cause of cancer death in males. Similarly, cancer of the ovary is the second most common cancer of the female reproductive organs and the fourth most common cause of cancer death among females. With few exceptions, metastatic disease from carcinoma is fatal. Even if patients survive their primary cancers, recurrence or metastases are common.

It is widely recognized that simple and rapid tests for solid cancers or tumors have considerable clinical potential. Not only can such tests be used for the early diagnosis of cancer but they also allow the detection of tumor recurrence following surgery and chemotherapy. A number of cancer-specific blood tests have been developed which depend upon the detection of tumor-specific antigens in the circulation (Catalona, W. J., et al., 1991, “Measurement of prostate-specific antigen in serum as a screening test for prostate cancer”, N. Engl. J. Med. 324, 1156-1161; Barrenetxea, G., et al., 1998, “Use of serum tumor markers for the diagnosis and follow-up of breast cancer”, Oncology, 55, 447-449; Cairns, P., and Sidreansky, D., 1999, “Molecular methods for the diagnosis of cancer”. Biochim. Biophys. Acta. 1423, C 11 -C 18).

Most ovarian cancers are thought to arise from the ovarian surface of epithelium (OSE). Epithelial ovarian cancer is seldom encountered in women less than 35 years of age. Its incidence increases sharply with advancing age and peaks at ages 75 to 80, with the median age being 60 years. The single most important known risk factor is a strong familial history of breast or ovarian cancer. To date, little is known about the structure and function of the OSE cells. It is known that the OSE is highly dynamic tissue that undergoes morphogenic changes, and has proliferative properties sufficient to cover the ovulatory site following ovulation. Morphological and histochemical studies suggest that the OSE has secretory, endocytotic and transport functions which are hormonally-controlled (Blaustein and Lee, Oncol. 8, 34-43, 1979; Nicosia and Johnson, Int. J. Gynecol. Pathol., 3, 249-260, 1983; Papadaki and Beilby, J. Cell Sci. 8, 445-464, 1971; Anderson et al., J. Morphol., 150, 135-164, 1976).

Ovarian cancers are not readily detectable by diagnostic techniques (Siemens et al., J. Cell. Physiol., 134: 347-356, 1988). In fact, the diagnosis of carcinoma of the ovary is generally only possible when the disease has progressed to a late stage of development. A number of proteinaceous ovarian tumor markers were evaluated several years ago, however these were found to be non-specific, and determined to be of low value as markers for primary ovarian cancer (Kudlacek et al., Gyn. Onc. 35, 323-329, 1989; Rustin et al., J. Clin. Onc., 7, 1667-1671, 1989; Sevelda et al., Am. J. Obstet. Gynecol., 161, 1213-1216, 1989; Omar et al., Tumor Biol., 10, 316-323, 1989). Several monoclonal antibodies were also shown to react with ovarian tumor associated antigens, however they were not specific for ovarian cancer and merely recognize determinants associated with high molecular weight mucin-like glycoproteins (Kenemans et al., Eur. J. Obstet. Gynecol. Repod. Biol. 29, 207-218, 1989; McDuffy, Ann. Clin. Biochem., 26, 379-387, 1989). More recently, oncogenes associated with ovarian cancers have been identified, including HER-21neu (c-erbB-2) which is over-expressed in one-third of ovarian cancers (U.S. Ser. No. 6,075,122 by Cheever et al., issued Jun. 13, 2000), the fms oncogene, and abnormalities in the p53 gene, which are seen in about half of ovarian cancers. Oncogene markers are not generally amenable to rapid and simple diagnosis of ovarian cancer, because they may be limited to cancer cell tissues and do not necessarily appear in mestases or in the circulation.

Recently, a GPCR designated as PHOR-1 was identified as having utility in the early detection of prostate cancer (see International Patent Publication No. WO01/25434, Apr. 12, 2001). Expression of PHOR-1 is localized to the prostate gland of healthy individuals, however is up-regulated in prostate tumors and can also be detected in tumors of the kidney, uterus, cervix, stomach and rectum. No other correlation has been recognized between GPCR polypeptide expression and cancer.

Whilst previously identified markers for carcinomas of the lung, prostate, breast, colon, pancreas, and ovary have facilitated efforts to diagnose and treat these serious diseases, there is a clear need for the identification of additional markers and therapeutic targets. The identification of tumor markers that are amenable to the early-stage detection of localized tumors is critical for more effective management of carcinomas of the lung, prostate, breast, colon, pancreas, and ovary.

### SUMMARY OF THE INVENTION

The present invention provides a novel GPCR-encoding gene, designated GPR56-1 or TSR32 (SEQ ID NO: 1). The structure of the encoded GPR56-1 polypeptide (i.e. SEQ ID NO: 2) is characteristic of known GPCR polypeptides of the secretin sub-family. The encoded polypeptide comprises about 693 amino acid residues in length (SEQ ID NO: 2) and includes the N-terminal sequence MTPQSLLQTT (SEQ ID NO: 20).

Homology searching has identified three additional human genes having at least about 95% identity, and more particularly about 99% identity to the GPR56-1 nucleotide sequence. These alleles are designated herein as GPR56-2, GPR56-3, and GPR56-4, respectively. At the protein level, the amino acid sequences of GPR56-1 (SEQ ID NO: 2) and GPR56-3 (SEQ ID NO: 6) are 100% identical, and GPR56-2 (SEQ ID NO: 4) differs from this isoform only by the substitution of Gln306 for His306. In contrast, the amino acid sequence of GPR56-4 (SEQ ID NO: 8) comprises a six amino acid deletion at positions 430-435 relative to the other two isoforms. These data indicate the existence of a small multigene family encoding GPR56 in humans.

Homology searching has also revealed a murine homologue (SEQ ID NO: 9) having about 80% identity to the nucleotide sequence encoding human GPR56-1. At the protein level the murine and human GPR56 polypeptides are about 80% identical over their entire lengths. The occurrence of long amino acid stretches that are highly conserved between the human and murine polypeptides indicates that these polypeptides are antigenically cross-reactive. Additionally, the high conservation between the human and murine nucleotide sequences is indicative that they cross-hybridize. This means that the murine GPR56-encoding nucleotide sequence can be used to detect the human GPR56-encoding genes. Similarly, the complement of the murine GPR56-encoding gene can be used to detect RNA encoding human GPR56 isoforms.

The present invention clearly encompasses any plasmids or expression vectors, including any viral vectors, comprising the nucleic acid described herein. Such vectors may be introduced into suitable host cells, such as, for example, bacterial cells, yeast cells, insect cells, or mammalian cells, for the purposes of expressing a recombinant GPR56 polypeptide or a functional fragment thereof, in particular an immunogenic peptide fragment.

The present invention further provides a method of producing a GPR56 polypeptide or a functional fragment thereof, said method comprising culturing a host cell comprising the nucleic acid of the invention in an expressible format under conditions sufficient for expression to occur and then recovering the expressed polypeptide. Preferably, the expressed polypeptide is directed to the cell surface. Preferably, the recovered polypeptide is rendered substantially free of conspecific proteins using known protein isolation/purification techniques. Preferred cell lines for this purpose are insect or mammalian cells. Baculovirus cell expression systems using Sf9 or Sf21 cells, or vaccinia virus expression systems using COS cells, CHO cells, or HEK 293 cells, are especially preferred for the synthesis of GPCR polypeptides.

Expression analyses using GeneChip microarray hybridization technology indicates that human GPR56 gene expression is up-regulated in patients suffering from ovarian cancer, including those subjects having metastases in the omentum or other tissues. At the RNA level, expression of GPR56 in ovarian tumor samples from both early and advanced ovarian cancer patients was up-regulated several-fold in the ovary and omentum, when compared to the level of expression detected in the ovary and omentum, respectively, of healthy subjects, indicating that GPR56 is a useful cancer marker, particularly for the detection of ovarian cancer and metastases thereof, and preferably for the early detection of ovarian cancer and metastases thereof. The actual enhancement of expression that was detected is much higher than for other known markers of ovarian cancer, making the instant invention particularly useful in terms of providing a definitive diagnostic or prognostic assay, since there is a significant distinction between GPR56 levels in diseased tissue and those of normal or healthy subjects. Moreover, the ability to detect ovarian cancer by measuring GPR56 expression in the omentum of a subject indicates that the present invention is not limited by the source of tissue used for the diagnosis of ovarian cancer. GPR56 expression was also detectable in prostate tumor samples.

The present invention clearly encompasses nucleic acid-based methods and protein-based methods for diagnosing cancer in humans and other mammals.

Those skilled in the art will be aware that as a carcinoma progresses, metastases occur in organs and tissues outside the site of the primary tumor. For example, in the case of ovarian cancer, metastases commonly appear in a tissue selected from the group consisting of omentum, abdominal fluid, lymph nodes, lung, liver, brain, and bone. The present inventors have found that GPR56 expression is useful for detecting any stage of progression of ovarian cancer, including early stages of the disease and metastases outside the ovary. Accordingly, the term “ovarian cancer” as used herein shall be taken to include an early or developed tumor of the ovary and any metastases outside the ovary that occurs in a subject having a primary tumor of the ovary.

As used herein, the term “diagnosis”, and variants thereof, such as, but not limited to “diagnose”, “diagnosed” or “diagnosing” shall not be limited to a primary diagnosis of a clinical state, however should be taken to include any primary diagnosis or prognosis of a clinical state. For example, the “diagnostic assay” formats described herein are equally relevant to assessing the remission of a patient, or monitoring disease recurrence, or tumor recurrence, such as following surgery or chemotherapy, or determining the appearance of metastases of a primary tumor. All such uses of the assays described herein are encompassed by the present invention.

Expression analyses using GeneChip microarray hybridization technology also indicate that human GPR56 gene expression on the surface of T cells is restricted to a sub-population of T cells that lack the CCR7 receptor (i.e. CCR7⁻ T cells). These data indicate that GPR56 is also a useful marker for detecting effector memory T cells that are involved in expressing receptors for migration to sites of inflammation and that possess immediate effector function. Such effector memory T cells contrast with the role of generally quiescent CCR7⁺ central memory T cells that express receptors for homing to lymph nodes and that lack immediate effector function. The detection of GPR56 expression on T cells is thus indicative of T cell activation in a sample.

Accordingly, the level of GPR56 expression, at either the RNA level or the protein level, can be used in a number of applications. Those applications include, for example, the identification of an effector memory T cell in a sample and the determination of an activated memory T cell count. Addtionally, GPR56 can be used to determine the infection status of an individual, and to determine whether or not an individual has been re-infected with an infectious agent, such as a bacterium or viral agent. Additionally, GPR56 expression can be used to diagnose inflammatory disease, or as a prognostic to monitor the progress of a disease state, such as, for example, a cancer, inflammatory disease, or chronic infection.

Based upon the nucleotide and amino acid sequence comparisons described herein, the present invention provides nucleic acid-based assays and immunoassays for the detection of cancer and/or effector memory T cells and/or T cell activation and/or diagnosing an inflammatory disorder which involves T cell activation.

More particularly, the nucleic acid-based assays described herein rely upon the detection or relative quantification of RNA levels in samples using probes of at least about 20 nucleotides in length that hybridize specifically to RNA encoding the GPR56 polypeptide, or alternatively, amplify cDNA from RNA encoding the GPR56 polypeptide. Such probes are derived from unique regions of any one or more of the GPR56-encoding genes described herein, such as, for example, any 20 contiguous nucleotides within residues 131-1400, 1423-2239, or 2264-2282 of SEQ ID NO: 1 or the protein-encoding region of SEQ ID NO: 1 or a complementary nucleotide sequence thereto or identical sequence in any other mammalian GPR56-encoding gene. The use of full-length antisense cDNA or cRNA derived from any one of SEQ ID NOS: 1, 3, 5, 7, or 9 is also encompassed by the present invention. Conveniently, any hybridization assay format can be used to detect GPR56-encoding RNA in samples, such as, for example, high-throughput screening using microarray technology, or conventional northern hybridization or reverse transcription polymerase chain reaction (i.e. RT-PCR). In situ localization can also be employed using histology specimens. Particularly preferred probes exemplified herein are those oligonucleotides having the nucleotide sequences set forth in any one of SEQ ID NOS: 11-19. Additional probes for use in these assays, including any allele-specific probes that selectively hybridize to one or more GPR56-encoding alleles, are not to be excluded and are readily identified by those skilled in the art based upon the nucleotide sequences, and the amino acid sequence alignment, provided herein.

The present invention clearly encompasses any nucleic acid probes or primers, including any synthetic oligonucleotides, suitable for use in the assays described herein.

In another embodiment, the nucleic acid encodes an antisense nucleic acid which can hybridize with a second nucleic acid encoding a GPR56 protein and which, when introduced into cells, can inhibit the expression of the GPR56 polypeptide in a cell, tissue, organ, or whole organism.

In another embodiment, the nucleic acid encodes an interfering RNA that can inhibit the expression of the GPR56 polypeptide in a cell, tissue, organ, or whole organism. By “interfering RNA” means an RNA molecule having a region of self-complementarity and/or capable of forming a hairpin loop structure in a cell, wherein said region of self-complementarity comprises a nucleotide sequence of at least about 20 contiguous nucleotides in length from a sequence having at least about 80% identity to SEQ ID NO: 1 or a complementary sequence thereto.

The immunoassays described herein utilize antibodies, including monoclonal and polyclonal antibodies, or a Fab fragment, F(ab′)₂ fragment, or scFv fragment, that binds to a unique peptide region comprising at least about 5-10 contiguous amino acid residues of a human GPR56 polypeptide. Homology searching indicates that most peptide regions of 5-10 amino acids in length from the human GPR56 polypeptide are unique, optionally excluding residues 414-419. Regions of the murine GPR56 polypeptide that are highly conserved with the human sequence (eg. any peptide comprising at least about 5-10 contiguous amino acid residues of SEQ ID NO: 10 that is identical to the corresponding region of SEQ ID NOS: 2, 4, 6, or 8, as shown in FIG. 1) are particularly useful for preparing antibodies against human GPR56. Isoform-specific amino acid sequences (eg. Amino acids 430-435 of SEQ ID NO: 2) are also readily derived from the alignment provided in FIG. 1, and these are used conveniently to prepare antibodies that detect specific GPR56 isoforms.

The present invention further encompasses any synthetic or recombinant peptides, or antibodies suitable for use in the assays described herein.

Antibodies or fragments thereof are useful in therapeutic, diagnostic and research applications, including the purification and study of the receptor proteins, identification of cells expressing surface receptor, and sorting or counting of cells. Thus, the present invention encompasses use of an antibody or fragment thereof described herein (e.g., monoclonal antibodies or an antigen-binding fragment thereof) in therapy, including prophylaxis, or diagnosis, and use of such antibodies or fragments for the manufacture of a medicament for use in treatment of diseases or conditions as described herein.

Also encompassed by the present invention are methods of identifying ligands of the GPR56 receptor polypeptide, such as, for example, inhibitors or antagonists, or alternatively, agonists of GPR56 receptor function. In one embodiment, suitable host cells that have been engineered to express GPR56 or a GPR56 homolog encoded by nucleic acid having at least about 80% identity to SEQ ID NO: 1 are are used in an assay to identify and assess the efficacy of ligands, agonists or antagonists of GPR56 function. Such cells are also useful in assessing the function of the expressed GPR56 protein or homolog.

According to the present invention, ligands, agonists, or antagonists of GPR56 function are identified in a suitable assay, and further assessed for their therapeutic efficacy. Antagonists of GPR56 are used to inhibit (ie. reduce or diminish or prevent) GPR56-mediated effects in cells, such as, for example, any hypoproliferative disease, inflammatory state, inflammation, or cancer. Alternatively, ligands and/or agonists of GPR56 are useful for inducing or enhancing GPR56-mediated effects in cells.

Accordingly, a further aspect of the present invention provides a method of treating a hypoproliferative disease, such as, for example, cancer, hyperimmune response, inflammatory disorder (eg. rheumatoid arthritis), autoimmune disease, or graft rejection, comprising administering an antagonist of GPR56 function to an individual (e.g., a mammal) for a time and under conditions sufficient to reduce or prevent GPR56 activity in said individual, thereby reducing or preventing one or more GPR56-mediated effects. Preferably, the antagonist comprises nucleic acid, such as, for example, antisense nucleic acid, a ribozyme, or nucleic acid that forms a triple helical structure, capable of reducing GPR56 expression in a cell of the individual. As will be known to those skilled in the art, the expression can be reduced at the RNA level or the protein level. Accordingly, antibodies that bind GPR56 and inhibit its activity are also useful in this context.

The present invention further provides a method of agonising or otherwise enhancing GPR56 activity in an individual comprising administering a GPR56 ligand or GPR56 agonist to said individual for a time and under conditions sufficient to enhance GPR56 activity. This embodiment of the invention clearly provides a new approach to selective enhancement of leukocyte activity, which is useful, for example, in the treatment of infectious diseases, particularly enhancing a response to re-infection by bacterial or viral pathogens.

### BRIEF DESCRIPTION OF THE DRAWINGS

FIGS. 1a-1k are a schematic representation of an alignment between the amino acid sequences of four human GPR56 polypeptides designated GPR56-1 (SEQ ID NO: 2), GPR56-2 (SEQ ID NO: 4), GPR56-3 (SEQ ID NO: 6) and GPR56-4 (SEQ ID NO: 8), and a single murine GPR56 polypeptide (SEQ ID NO: 10). Amino acid residues that differ from the amino acid sequence of GPR56-1 (SEQ ID NO: 2) are indicated in bold typeface. Numbering below each block of five sequences indicates the amino acid residue number relative to the full-length sequence set forth in SEQ ID NO: 2.

FIGS. 2a-2d are a graphical representation of an RNA-DNA hybridization showing relative expression of human GPR56 RNA in normal and tumorigenic ovary or omentum samples from human subjects. Subjects included 6 patients diagnosed as having borderline ovary cancer using conventional screens (columns numbered 1 through 6); 34 patients definitively diagnosed as having ovarian cancer, using conventional screens (columns numbered 7 through 44); and 11 ovarian cancer patients diagnosed with metastases or secondary cancers of the omentum using conventional screens (columns numbered 45 through 55). Control samples comprised non-cancerous ovary tissue (columns numbered 56 through 59) and non-cancerous omentum (column 60). The x-axis indicates sample number. The abscissa indicates relative GPR56 RNA expression. Numbers at the top of each column indicate the relative expression for each sample. Data indicate a 6- to 7-fold enhancement of GPR56 expression in early and advanced cancers of the ovary, and/or metastases of the omentum (ie. peritoneum connecting the stomach and other abdominal organs).

### DETAILED DESCRIPTION OF THE INVENTION

One aspect of the present invention provides an isolated nucleic acid molecule comprising a nucleotide sequence that encodes a G protein-coupled receptor (GPCR or GPR) polypeptide, and preferably a GPR56 polypeptide or an immunologically active derivative thereof.

As used herein, the term “nucleic acid” shall be taken to mean any single-stranded or double-stranded RNA, DNA, cDNA, cRNA, or synthetic oligonucleotide, or alternatively, an analog of RNA, DNA, cDNA, cRNA, or a synthetic oligonucleotide. “Nucleic acid” also includes any genomic gene equivalents of a cDNA molecule.

In a preferred embodiment, the isolated nucleic acid of the invention is from humans (ie. it encodes a human GPR56 polypeptide).

As used herein, the term “GPR56 polypeptide” shall be taken to mean a GPR polypeptide of mammals having at least about 80% identity at the amino acid level to the amino acid sequence set forth in SEQ ID NO: 2. Preferably, the percentage identity to SEQ ID NO: 2 is at least about 90%, more preferably at least about 95%, or at least about 99%.

In determining whether or not two amino acid sequences fall within these defined percentage identity limits, those skilled in the art will be aware that it is necessary to conduct a side-by-side comparison of amino acid sequences. In such comparisons or alignments, differences will arise in the positioning of non-identical amino acid residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using the GAP program of the Computer Genetics Group, Inc., University Research Park, Madison, Wis., United States of America (Devereaux et al, Nucl. Acids Res. 12, 387-395,1984), which utilizes the algorithm of Needleman and Wunsch J. Mol. Biol. 48, 443-453, 1970, or alternatively, the CLUSTAL W algorithm of Thompson et al., Nucl. Acids Res. 22, 4673-4680, 1994, for multiple alignments, to maximize the number of identical/similar amino acids and to minimize the number and/or length of sequence gaps in the alignment.

Alternatively, or in addition, an isolated nucleic acid encoding a GPR56 polypeptide hybridizes under high stringency conditions to a sequence that is complementary to SEQ ID NO: 1. For the purposes of defining the level of stringency, a high stringency hybridization is achieved using a hybridization buffer and/or a wash solution comprising the following:

(i) a salt concentration that is equivalent to 0.1×SSC-0.2×SSC buffer or lower salt concentration;

(ii) a detergent concentration equivalent to 0.1% (w/v) SDS or higher; and

(iii) an incubation temperature of 55° C. or higher.

Conditions for specifically hybridizing nucleic acid, and conditions for washing to remove non-specific hybridizing nucleic acid, are well understood by those skilled in the art. For the purposes of further clarification only, reference to the parameters affecting hybridization between nucleic acid molecules is found in Ausubel et al. (Current Protocols in Molecular Biology, Wiley Interscience, ISBN 047150338, 1992), which is herein incorporated by reference.

Preferably, the isolated nucleic acid is expressed in hematopoietic cells, including stem cells, epithelial cells, and T cells and/or in one or more tissues selected from the group consisting of serum, abdominal fluid, lymph, lung, prostate, omentum, ovary, liver, placenta, and brain, as determined by the appearance of RNA encoding said polypeptide in those tissues.

Preferably, the isolated nucleic acid of the invention is also expressed in a range of cancer cells, such as, for example, in carcinomas of the lung, prostate, breast, colon, pancreas, placenta, omentum or ovary, and in cells of brain anaplastic oligodendrogliomas, as determined by the appearance of RNA encoding said polypeptide in those cells.

In the present context, the term “cancer cell” includes any biological specimen or sample comprising a cancer cell irrespective of its degree of isolation or purity, such as, for example, tissues, organs, cell lines, bodily fluids, or histology specimens that comprise a cell in the early stages of transformation or having been transformed. Bodily fluids shall be taken to include whole blood, serum, peripheral blood mononuclear cells (PBMC), or buffy coat fraction.

As the present invention is particularly useful for the early detection of cancer, the definition of “cancer cell” is not to be limited by the stage of a cancer in the subject from which said cancer cell is derived (ie. whether or not the patient is in remission or undergoing disease recurrence or whether or not the cancer is a primary tumor or the consequence of metastases). Nor is the term “cancer cell” to be limited by the stage of the cell cycle of said cancer cell.

Even more preferably, the isolated nucleic acid is expressed at elevated levels in cancer cells compared to non-cancer cells, as detected by measuring the level of GPR56 RNA or GPR56 polypeptide. In a particularly preferred embodiment, the isolated nucleic acid of the invention is expressed at an elevated level in ovarian cancer cells, such as, but not limited to, cancerous OSE cells, and metastases thereof, such as, for example, omentum, abdominal fluid, lymph nodes, lung, liver, brain, or bone.

In an alternative preferred embodiment, the isolated nucleic acid is expressed on the surface of effector memory T cells, wherein it is capable of being used for providing a number of beneficial data sets for an individual, such as, for example, immune status, infection status, response to re-infection, activated memory T cell count, inflammation status, or inflammatory disease state. As used herein, the term “effector memory T cell” shall be taken to mean a memory T cell that expresses a receptor to facilitate its migration to a site of inflammation and/or that possesses immediate effector function and/or that lacks a functional CCR7 receptor.

In a particularly preferred embodiment, the isolated nucleic acid of the present invention comprises a nucleotide sequence selected from the group consisting of:

(i) the nucleotide sequence set forth in SEQ ID NO: 1;

(ii) nucleotide residues 163 to 2241 of SEQ ID NO: 1;

(iii) a nucleotide sequence that encodes the amino acid sequence set forth in SEQ ID NO: 2; and

(iv) a sequence that is complementary to any one of (i) to (iii).

The term “immunologically active derivative” shall be taken to mean any peptide fragment of a GPR56 polypeptide that is of a sufficient length and/or sufficiently antigenic to: (i) facilitate the production of antibodies that can detect GPR56 in samples; and/or (ii) bind to antibodies against a GPR56 polypeptide.

Such “derivatives”, or their functional equivalents, may be generated by several means known to those skilled in the art, such as, for example:

(i) digestion of a GPR56 polypeptide comprising the amino acid sequence of SEQ ID NO: 2 or an immunologically active derivative thereof or a functional equivalent thereof, using a reagent such as, for example, cyanogen bromide, S-ethyltrifluorothioacetate, trypsin, chymotrypsin, pepsin, or thermolysin;

(ii) chemical peptide synthesis of a peptide comprising at least about 5-10 contiguous amino acids in length of SEQ ID NO: 2 or a functionally equivalent peptide thereto, using art-recognized techniques, such as, for example, Fmoc chemistry (reviewed by Fields (ed), Methods. Enzymol. 289, Academic Press, 1997 (whole of volume); Hecht, S. M. (ed) Bioorganic Chemistry: Peptides and Proteins, Oxford University Press: New York, ISBN 0-19-508468-3, 1998; Mayo, TIBTECH 18, 212-217, 2000);

(iii) by recombinant expression of a nucleic acid fragment of the full-length protein-encoding region of SEQ ID NO: 1 or an equivalent thereof in a suitable cellular or cell-free expression system (see below); and

(iv) subjecting the nucleotide sequence of the full-length protein-encoding region of SEQ ID NO: 1 or a functional equivalent thereof to site-directed mutagenesis (reviewed by Hecht, S. M. (ed) Bioorganic Chemistry: Peptides and Proteins, Oxford University Press: New York, ISBN 0-19-508468-3, 1998), so as to produce single or multiple nucleotide substitutions, deletions and/or insertions that have minimal adverse effect on the antigenicity of the peptide encoded by the mutated sequence relative to the wild-type (non-mutant) sequence or the ability of said peptide to bind antibodies that recognize the full-length native GPR56 polypeptide.

For producing full-length polypeptides or immunologically active derivatives thereof by recombinant means, a protein-encoding region comprising at least about 15 contiguous nucleotides of the protein-encoding region of SEQ ID NO: 1, or an equivalent region from another GPR56-encoding gene, is placed in operable connection with a promoter or other regulatory sequence capable of regulating expression in a cell-free system or cellular system.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or external stimuli, or in a tissue-specific manner. In the present context, the term “promoter” is also used to describe a recombinant, synthetic or fusion molecule, or derivative which confers, activates or enhances the expression of a nucleic acid molecule to which it is operably connected, and which encodes the polypeptide or peptide fragment. Preferred promoters can contain additional copies of one or more specific regulatory elements to further enhance expression and/or to alter the spatial expression and/or temporal expression of the said nucleic acid molecule.

Placing a nucleic acid molecule under the regulatory control of, i.e., “in operable connection with”, a promoter sequence means positioning said molecule such that expression is controlled by the promoter sequence. Promoters are generally positioned 5′ (upstream) to the coding sequence that they control. To construct heterologous promoter/structural gene combinations, it is generally preferred to position the promoter at a distance from the gene transcription start site that is approximately the same as the distance between that promoter and the gene it controls in its natural setting, i.e., the gene from which the promoter is derived. Furthermore, the regulatory elements comprising a promoter are usually positioned within 2 kb of the start site of transcription of the gene. As is known in the art, some variation in this distance can be accommodated without loss of promoter function. Similarly, the preferred positioning of a regulatory sequence element with respect to a heterologous gene to be placed under its control is defined by the positioning of the element in its natural setting, i.e., the genes from which it is derived. Again, as is known in the art, some variation in this distance can also occur.

The prerequisite for producing intact polypeptides and peptides in bacteria such as E. coli is the use of a strong promoter with an effective ribosome binding site. Typical promoters suitable for expression in bacterial cells such as E. coli include, but are not limited to, the lacz promoter, temperature-sensitive λ_{L} or λ_{R} promoters, T7 promoter or the IPTG-inducible tac promoter. A number of other vector systems for expressing the nucleic acid molecule of the invention in E. coli are well-known in the art and are described, for example, in Ausubel et al (In: Current Protocols in Molecular Biology. Wiley Interscience, ISBN 047150338, 1987) or Sambrook et al (In: Molecular cloning. A laboratory manual, second edition, Cold Spring Harbor Laboratory, Cold Spring Harbor, N.Y., 1989). Numerous plasmids with suitable promoter sequences for expression in bacteria and efficient ribosome binding sites have been described, such as for example, pKC30 (λ_{L}: Shimatake and Rosenberg, Nature 292, 128, 1981); pKK173-3 (tac: Amann and Brosius, Gene 40, 183, 1985), pET-3 (T7: Studier and Moffat, J. Mol. Biol. 189, 113, 1986); the pBAD/TOPO or pBAD/Thio-TOPO series of vectors containing an arabinose-inducible promoter (Invitrogen, Carlsbad, Calif.), the latter of which is designed to also produce fusion proteins with thioredoxin to enhance solubility of the expressed protein; the pFLEX series of expression vectors (Pfizer Inc., CT, USA); or the pQE series of expression vectors (Qiagen, CA), amongst others.

Typical promoters suitable for expression in viruses of eukaryotic cells and eukaryotic cells include the SV40 late promoter, SV40 early promoter and cytomegalovirus (CMV) promoter, CMV IE (cytomegalovirus immediate early) promoter amongst others. Preferred vectors for expression in mammalian cells (eg. 293, COS, CHO, 293T cells) include, but are not limited to, the pcDNA vector suite supplied by Invitrogen, in particular pcDNA 3.1 myc-His-tag comprising the CMV promoter and encoding a C-terminal 6× His and MYC tag; and the retrovirus vector pSRαtkneo (Muller et al., Mol. Cell. Biol., 11, 1785, 1991). The vector pcDNA 3.1 myc-His (Invitrogen) is particularly preferred for expressing a secreted form of GPR56 or a derivative thereof in 293T cells, wherein the expressed peptide or protein can be purified free of conspecific proteins, using standard affinity techniques that employ a Nickel column to bind the protein via the His tag.

A wide range of additional host/vector systems suitable for expressing GPR56 polypeptides or immunological derivatives thereof are available publicly, and described, for example, in Sambrook et al (In: Molecular cloning. A laboratory manual, second edition, Cold Spring Harbor Laboratory, Cold Spring Harbor, N.Y., 1989).

Means for introducing the isolated nucleic acid molecule or a gene construct comprising same into a cell for expression are well-known to those skilled in the art. The technique used for a given organism depends on the known successful techniques. Means for introducing recombinant DNA into animal cells include microinjection, transfection mediated by DEAE-dextran, transfection mediated by liposomes such as by using lipofectamine (Gibco, MD, USA) and/or cellfectin (Gibco, MD, USA), PEG-mediated DNA uptake, electroporation and microparticle bombardment such as by using DNA-coated tungsten or gold particles (Agracetus Inc., WI, USA) amongst others.

For producing mutants, nucleotide insertion derivatives of the protein-encoding region of SEQ ID NO: 1 or an equivalent thereof are produced by making 5′ and 3′ terminal fusions, or by making intra-sequence insertions of single or multiple nucleotides or nucleotide analogues. Insertion nucleotide sequence variants are produced by introducing one or more nucleotides or nucleotide analogues into a predetermined site in the nucleotide sequence of said sequence, although random insertion is also possible with suitable screening of the resulting product being performed. Deletion variants are produced by removing one or more nucleotides from the nucleotide sequence. Substitutional nucleotide variants are produced by substituting at least one nucleotide in the sequence with a different nucleotide or a nucleotide analogue in its place, with the immunologically active derivative encoded therefor having an identical amino acid sequence to a derivative of SEQ ID NO: 2, or only a limited number of amino acid modifications that do not alter its antigenicity compared to the base derivative of SEQ ID NO: 2 or its ability to bind antibodies prepared against the base to a derivative of SEQ ID NO: 2. Such mutant derivatives will preferably have at least 80% identity with the base amino acid sequence from which they are derived (ie. the base derivative of SEQ ID NO: 2).

Preferred immunologically active derivatives of the full-length GPR56 polypeptide will comprise at least about 5-10 contiguous amino acids of the full-length amino acid sequence, more preferably at least about 10-20 contiguous amino acids in length, and even more preferably 20-30 contiguous amino acids in length.

For the purposes of producing derivatives using standard peptide synthesis techniques, such as, for example, Fmoc chemistry, a length not exceeding about 30-50 amino acids in length is preferred, as longer peptides are difficult to produce at high efficiency. Longer peptide fragments are readily achieved using recombinant DNA techniques wherein the peptide is expressed in a cell-free or cellular expression system comprising nucleic acid encoding the desired peptide fragment.

In view of the very high percentage identity between the amino acid sequences of the human GPR56 isoforms exemplified herein, any sufficiently antigenic region of at least about 5-10 amino acid residues in length derived from SEQ ID NO: 1, with the exception of amino acid residues 430-435 which are specific for isoforms GPR56-1, GPR56-2 and GPR56-3, can be used to prepare antibodies that bind generally to human GPR56 polypeptides. In fact, any of the exemplified full-length human GPR56 polypeptides are contemplated to immunologically cross-react. Accordingly, immunologically active derivatives of any one of SEQ ID NOS: 4, 6, or 8 are functionally equivalent to immunologically active derivatives of SEQ ID NO: 2, and thus encompassed by the present invention.

Preferably, amino acid residues 430-435 of SEQ ID NO: 1 are used for preparing isoform-specific antibodies that bind to human GPR56-1, GPR56-2 and GPR56-3, but not to human GPR56-4 (or to murine GPR56).

Moreover, the high conservation between the amino acid sequences of the murine and human GPR56 polypeptides exemplified herein also indicates that it is possible to use specific regions of the murine GPR56 polypeptide to produce immunologically active derivatives of SEQ ID NO: 10 that are functionally equivalent to immunologically active derivatives of SEQ ID NO: 2. Such equivalents are clearly encompassed by the present invention.

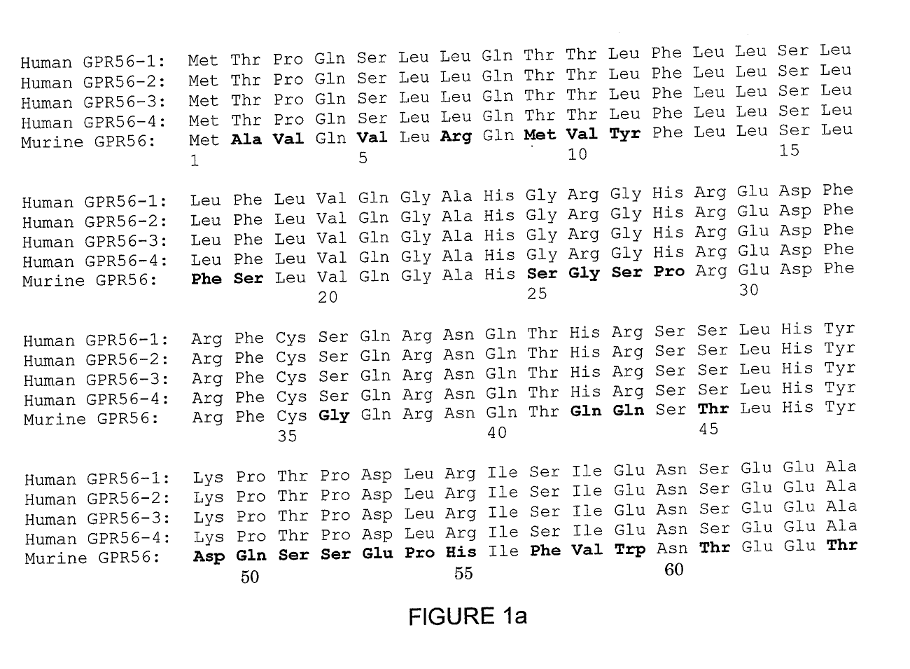

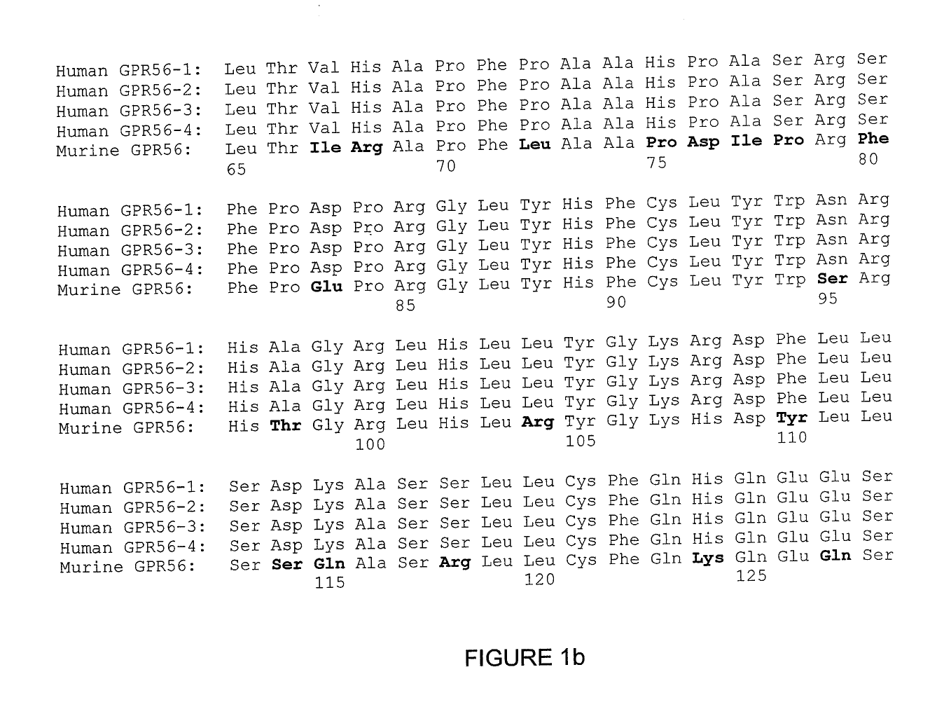

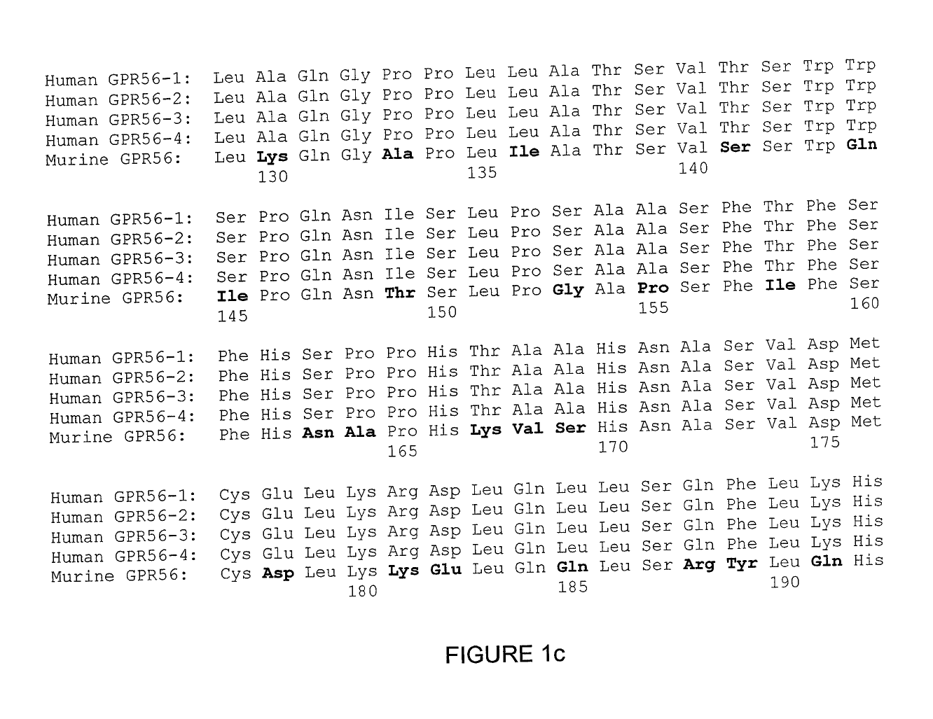

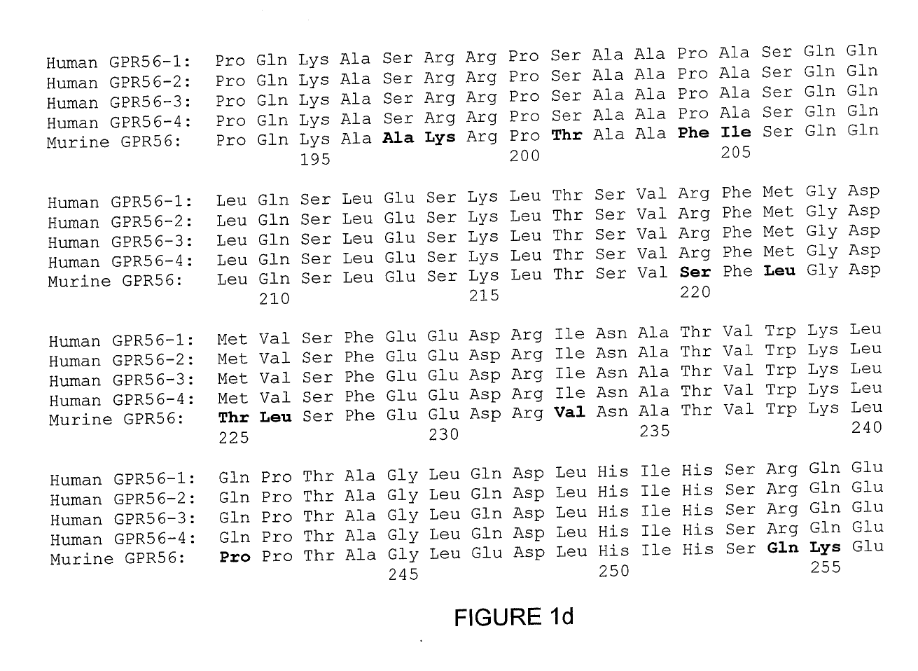

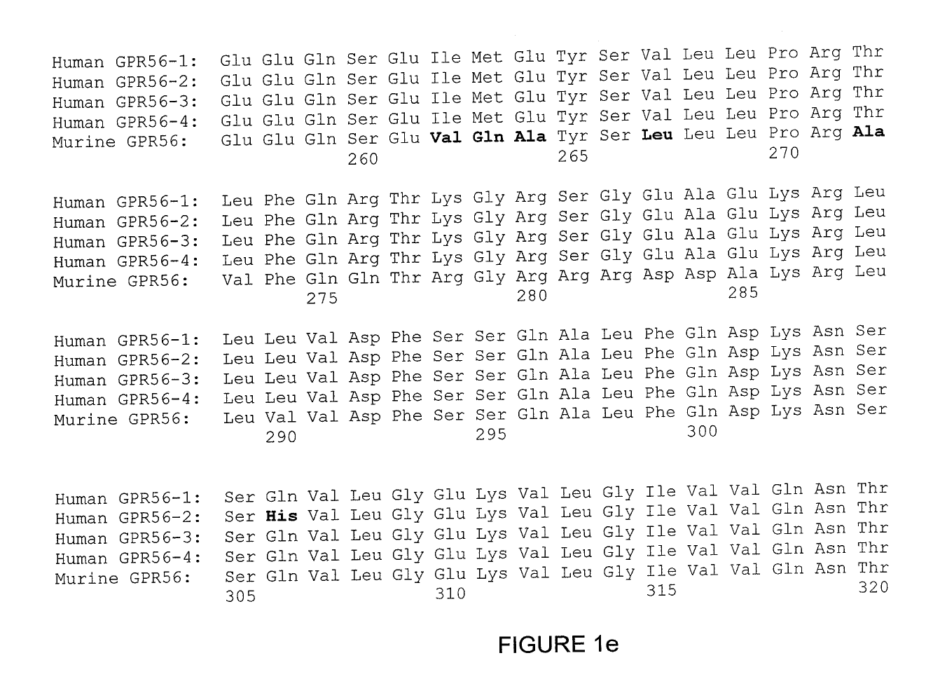

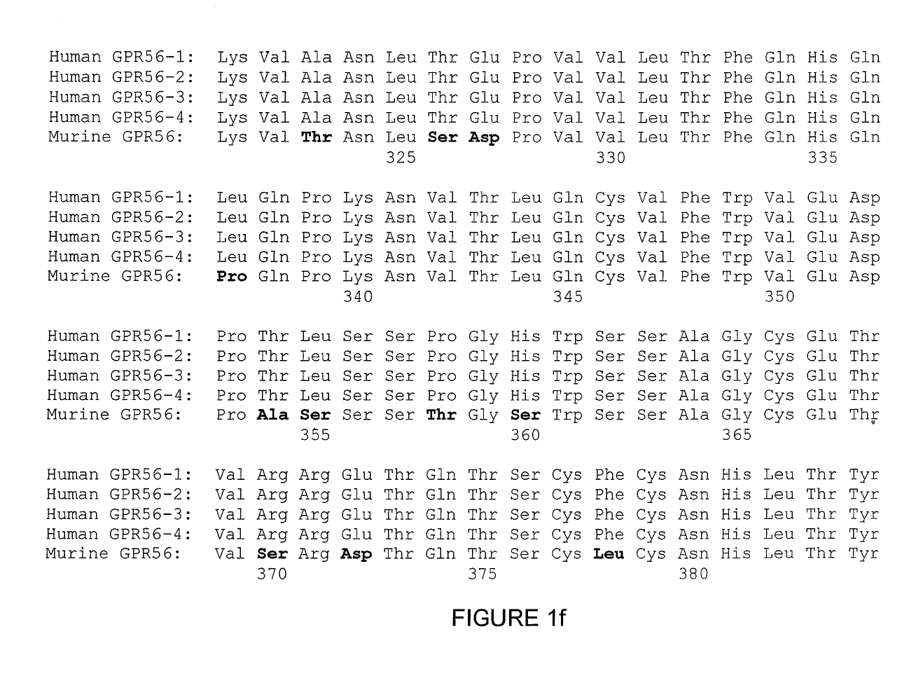

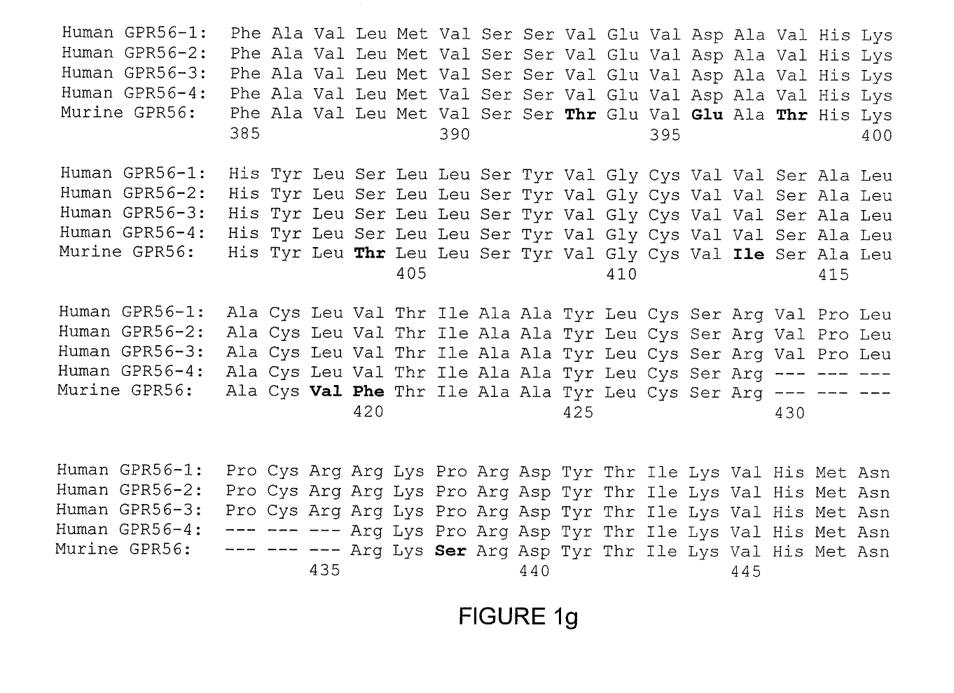

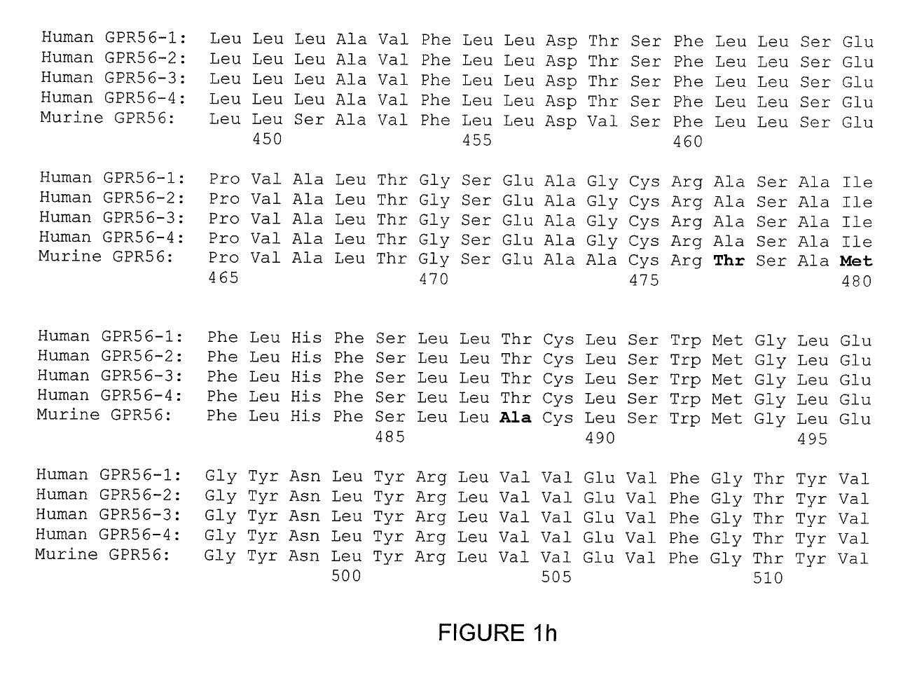

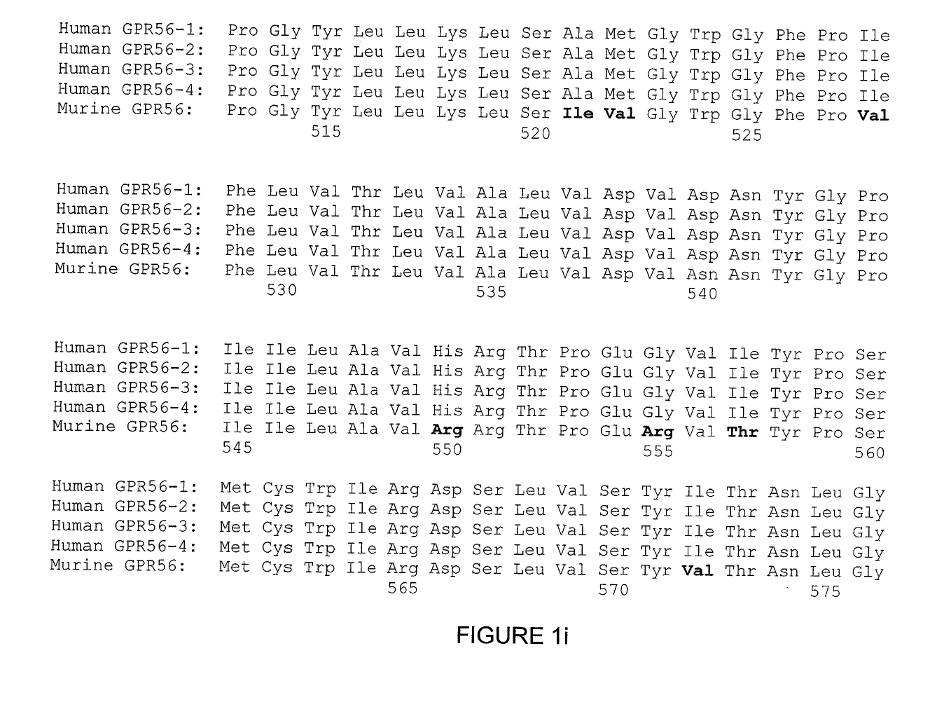

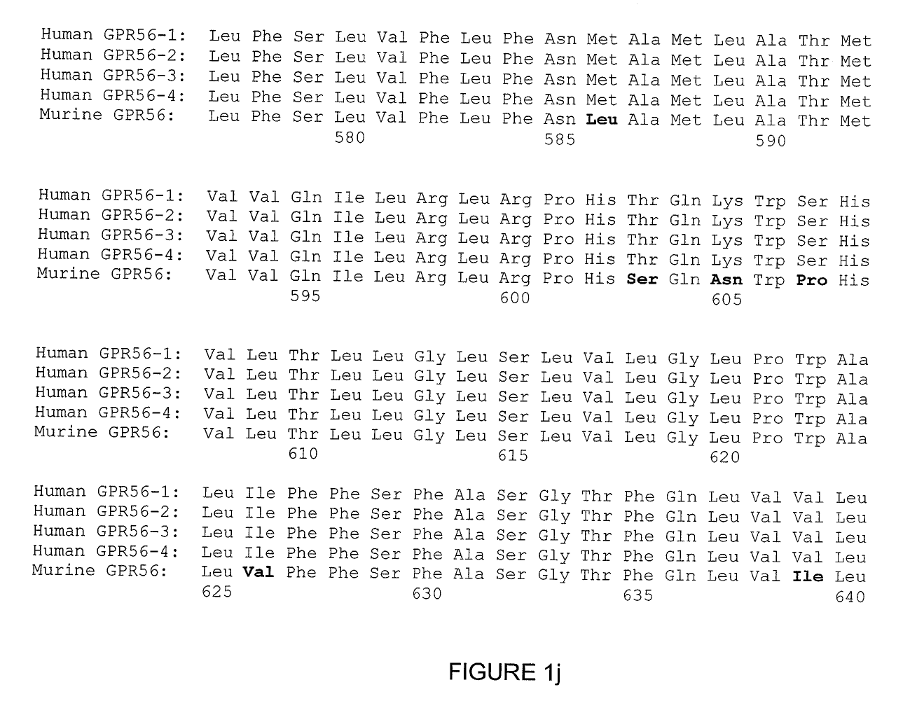

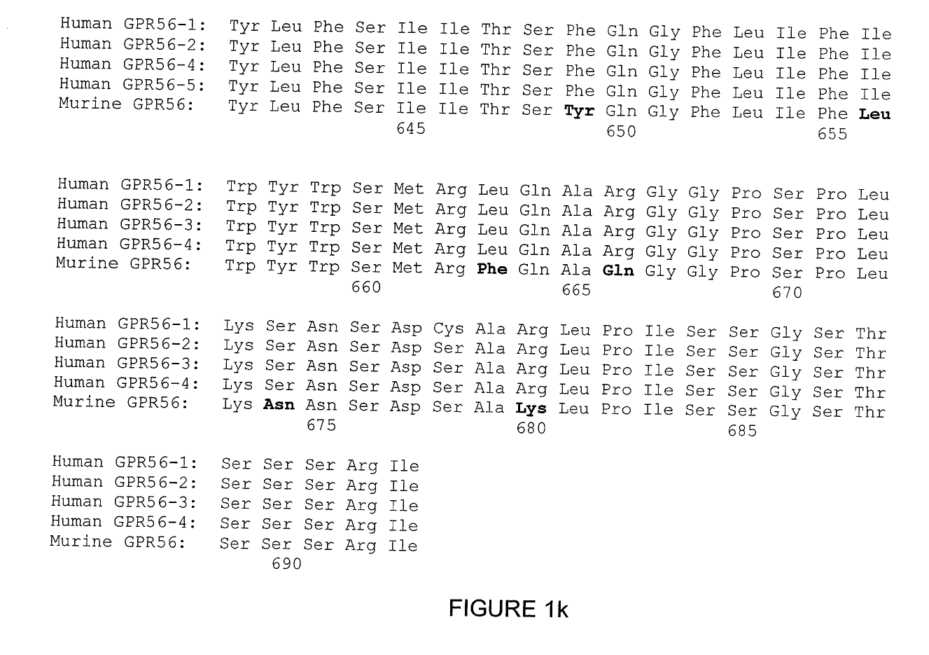

Preferred derivatives of human GPR56 (SEQ ID NO: 2) that are absolutely conserved in the amino acid sequences of mammalian GPR56, as shown in FIG. 1, will comprise at least about 5 contiguous amino acid residues in length of a sequence selected from the group consisting of:

(i) amino acid residues 12-16 of SEQ ID NO: 2;

(ii) amino acid residues 19-24 of SEQ ID NO: 2;

(iii) amino acid residues 29-35 of SEQ ID NO: 2;

(iv) amino acid residues 37-41 of SEQ ID NO: 2;

(v) amino acid residues 84-94 of SEQ ID NO: 2;

(vi) amino acid residues 99-103 of SEQ ID NO: 2;

(vii) amino acid residues 105-109 of SEQ ID NO: 2;

(viii) amino acid residues 119-123 of SEQ ID NO: 2;

(ix) amino acid residues 170-177 of SEQ ID NO: 2;

(x) amino acid residues 192-196 of SEQ ID NO: 2;

(xi) amino acid residues 206-219 of SEQ ID NO: 2;

(xii) amino acid residues 227-232 of SEQ ID NO: 2;

(xiii) amino acid residues 234-240 of SEQ ID NO: 2;

(xiv) amino acid residues 242-253 of SEQ ID NO: 2;

(xv) amino acid residues 256-261 of SEQ ID NO: 2;

(xvi) amino acid residues 273-305 of SEQ ID NO: 2;

(xvii) amino acid residues 307-322 of SEQ ID NO: 2;

(xviii) amino acid residues 328-336 of SEQ ID NO: 2;

(xix) amino acid residues 338-353 of SEQ ID NO: 2;

(xx) amino acid residues 361-369 of SEQ ID NO: 2;

(xxi) amino acid residues 373-377 of SEQ ID NO: 2;

(xxii) amino acid residues 379-392 of SEQ ID NO: 2;

(xxiii) amino acid residues 398-403 of SEQ ID NO: 2;

(xxiv) amino acid residues 405-412 of SEQ ID NO: 2;

(xxv) amino acid residues 414-418 of SEQ ID NO: 2;

(xxvi) amino acid residues 421-429 of SEQ ID NO: 2;

(xxvii) amino acid residues 439-475 of SEQ ID NO: 2;

(xxviii) amino acid residues 4481-486 of SEQ ID NO: 2;

(xxix) amino acid residues 488-519 of SEQ ID NO: 2;

(xxx) amino acid residues 522-526 of SEQ ID NO: 2;

(xxxi) amino acid residues 528-548 of SEQ ID NO: 2;

(xxxii) amino acid residues 557-570 of SEQ ID NO: 2;

(xxxiii) amino acid residues 572-584 of SEQ ID NO: 2;

(xxxiv) amino acid residues 586-601 of SEQ ID NO: 2;

(xxxv) amino acid residues 607-624 of SEQ ID NO: 2;

(xxxvi) amino acid residues 626-637 of SEQ ID NO: 2;

(xxxvii) amino acid residues 639-647 of SEQ ID NO: 2;

(xxxviii) amino acid residues 649-654 of SEQ ID NO: 2;

(xxxix) amino acid residues 656-661 of SEQ ID NO: 2;

(xl) amino acid residues 666-672 of SEQ ID NO: 2;

(xli) amino acid residues 674-678 of SEQ ID NO: 2; and

(xlii) amino acid residues 680-693 of SEQ ID NO: 2.

The present invention clearly extends to any analogs of an isolated nucleic acid encoding GPR56 or an immunologically active derivative of GPR56. By “analog” is meant nucleic acid that encodes GPR56 or a derivative of GPR56 and includes one or more nucleotide or non-nucleotide substituents not normally present in said isolated nucleic acid, such as, for example a carbohydrate, radiochemical, fluorescent molecule, biotin, DIG, alkaline phosphatase, horseradish peroxidase, or other reporter molecule. Preferred reporter molecules include radioactively-labelled nucleotide triphosphates and biotinylated molecules. Analogs are generally produced to facilitate detection of the nucleic acid.

A second aspect of the present invention clearly extends to an isolated GPR polypeptide, and preferably a GPR56 polypeptide or an immunologically active derivative thereof.

In a particularly preferred embodiment, the isolated polypeptide of the invention is substantially free of conspecific proteins. Such purity can be assessed by standard procedures, such as, for example, SDS/polyacrylamide gel electrophoresis, 2-dimensional gene electrophoresis, chromatography, amino acid composition analysis, or amino acid sequence analysis.

To produce isolated GPR56 polypeptides or fragments, standard protein purification techniques may be employed. For example, gel filtration, ion exchange chromatography, reverse phase chromatography, or affinity chromatography, or a combination of any one or more said procedures, may be used. High pressure and low pressure procedures can also be employed, such as, for example, FPLC, or HPLC. To isolate the full-length GPR56 polypeptide, or a fragment thereof comprising more than about 50-100 amino acids in length, it is particularly preferred to express the polypeptide in a suitable cellular expression system in combination with a suitable affinity tag, such as a 6× His tag, and to purify the polypeptide using an affinity step that bonds it via the tag (supra). Optionally, the tag may then be cleaved from the expressed polypeptide.

Alternatively, for short immunologically active derivatives of the full-length polypeptide, preferably those peptide fragments comprising less than about 50 amino acids in length, chemical synthesis techniques are conveniently used. As will be known to those skilled in the art, such techniques may also produce contaminating peptides that are shorter than the desired peptide, in which case the desired peptide is conveniently purified using reverse phase and/or ion exchange chromatography procedures at high pressure (ie. HPLC or FPLC).

In a particularly preferred embodiment, the isolated polypeptide of the invention will comprise the amino acid sequence set forth in SEQ ID NO: 2 or an immunologically active derivative thereof.

A third aspect of the present invention provides a nucleic acid probe for detecting RNA encoding a GPR56 polypeptide in a sample.

A “nucleic acid probe” is any nucleic acid as hereinbefore defined that is useful for detecting RNA encoding a GPR56 polypeptide or a derivative or analog thereof in a sample. Nucleic acid probes can comprise inosine, adenine, guanine, thymidine, cytidine or uracil residues or functional analogues or derivatives thereof that are capable of being incorporated into a polynucleotide molecule, provided that the resulting probe or primer is capable of hybridizing under at least low stringency conditions to GPR56-encoding RNA or DNA.

Whilst the probes may comprise double-stranded or single-stranded nucleic acid, single-stranded probes are preferred because they do not require melting prior to use in hybridizations. On the other hand, longer probes are also preferred because they can be used at higher hybridization stringency than shorter probes and may produce lower background hybridization than shorter probes.

So far as shorter probes are concerned, single-stranded, chemically-synthesized oligonucleotide probes are particularly preferred by the present invention. To reduce the noise associated with the use of such probes during hybridization, the nucleotide sequence of the probe is carefully selected to maximize the Tm at which hybridizations can be performed, reduce non-specific hybridization, and to reduce self-hybridization. Such considerations may be particularly important for applications involving high throughput screening using microarray technology. In general, this means that the nucleotide sequence of an oligonucleotide probe is selected such that it is unique to GPR56 RNA or GPR56 protein-encoding sequence, has a low propensity to form secondary structure, low self-complementary, and is not highly A/T-rich.

The only requirement for the probes is that they cross-hybridize to nucleic acid encoding GPR56 or the complementary nucleotide sequence thereto and are sufficiently unique in sequence to generate high signal:noise ratios under specified hybridization conditions. As will be known to those skilled in the art, long nucleic acid probes are preferred because they tend to generate higher signal:noise ratios than shorter probes and/or the duplexes formed between longer molecules have higher melting temperatures (i.e. Tm values) than duplexes involving short probes. Accordingly, full-length DNA or RNA probes are contemplated by the present invention, as are specific probes comprising the sequence of the 3′-untranslated region or complementary thereto.

In a particularly preferred embodiment, the nucleotide sequence of an oligonucleotide probe has no detectable nucleotide sequence identity to a nucleotide sequence in a BLAST search (Altschul et al., J. Mol. Biol. 215, 403-410, 1990) or other database search, other than a sequence selected from the group consisting of: (a) a sequence encoding a human GPR56 polypeptide; (b) the 5′-untranslated region of a sequence encoding a human GPR56 polypeptide; (c) a 3′-untranslated region of a sequence encoding a human GPR56 polypeptide; and (d) an exon region of a sequence encoding a human GPR56 polypeptide.

Even more preferably, the nucleotide sequence of an oligonucleotide probe has the following properties:

(i) it comprises less than ten(10) A residues;

(ii) it comprises less than ten(10) T residues;

(iii) it comprises less than nine(9) C residues;

(iv) it comprises less than nine(9) G residues;

(v) it comprises less than seven(7) A residues in any window consisting of 8 nucleotides;

(vi) it comprises less than seven(7) T residues in any window consisting of 8 nucleotides;

(vii) it comprises less than eight(8) C residues in any window consisting of 8 nucleotides;

(viii) it comprises less than eight(8) G residues in any window consisting of 8 nucleotides;

(ix) it comprises less than six(6) consecutive A residues;

(x) it comprises less than six(6) consecutive T residues;

(xi) it comprises less than five(5) consecutive C residues; and

(xii) it comprises less than five(5) consecutive G residues.

Additionally, the self-complementarity of a nucleotide sequence can be determined by aligning the sequence with its reverse complement, wherein detectable regions of identity are indicative of potential self-complementarity. As will be known to those skilled in the art, such sequences may not necessarily form secondary structures during hybridization reaction, and, as a consequence, successfully identify a target nucleotide sequence. It is also known to those skilled in the art that, even where a sequence does form secondary structures during hybridization reactions, reaction conditions can be modified to reduce the adverse consequences of such structure formation. Accordingly, a potential for self-complementarity should not necessarily exclude a particular candidate oligonucleotide from selection. In cases where it is difficult to determine nucleotide sequences having no potential self-complementarity, the uniqueness of the sequence should outweigh a consideration of its potential for secondary structure formation.

Recommended pre-requisites for selecting oligonucleotide probes, particularly with respect to probes suitable for microarray technology, are described in detail by Lockhart et at., “Expression monitoring by hybridization to high-density oligonucleotide arrays”, Nature Biotech. 14, 1675-1680, 1996.

The nucleic acid probe may comprise a nucleotide sequence that is within the coding strand of the GPR56-encoding gene (ie. it is comprised within the nucleotide sequence of RNA encoding GPR56). Such “sense” probes are useful for detecting RNA encoding GPR56 by amplification procedures, such as, for example, polymerase chain reaction (PCR), and more preferably, quantitative PCR or reverse transcription polymerase chain reaction (RT-PCR). Alternatively, “sense” probes may be expressed to produce GPR56 polypeptides or immunologically active derivatives thereof that are useful for detecting the expressed GPR56 protein in samples.

Preferred sense probes for detecting RNA encoding GPR56 comprise a nucleotide sequence selected from the group consisting of:

(i) a nucleotide sequence having at least 80% identity to SEQ ID NO: 1;

(ii) a nucleotide sequence comprising nucleotide residues 131-1400 of SEQ ID NO: 1 or a sequence having 80% identity thereto;

(iii) a nucleotide sequence comprising nucleotide residues 1423-2239 of SEQ ID NO: 1 or a sequence having 80% identity thereto;

(iv) a nucleotide sequence comprising nucleotide residues 2264-2282 of SEQ ID NO: 1 or a sequence having 80% identity thereto;

(v) a nucleotide sequence comprising at least 20 contiguous nucleotides of any one of (i) through (iv); and

(vi) an analog of any one of (i) through (v) as herein before defined.

More particularly, a sense probe will comprise a nucleotide sequence selected from the group consisting of:

(i) the sequence set forth in SEQ ID NO: 11;

(ii) the sequence set forth in SEQ ID NO: 12; and

(iii) the sequence set forth in SEQ ID NO: 13,

Alternatively, within the antisense strand of said gene (i.e. it is complementary to RNA encoding GPR56). Such “antisense” probes are useful for directly hybridizing to RNA encoding GPR56, or alternatively, for detecting RNA encoding GPR56 by amplification, as described supra (eg. quantitative PCR or RT-PCR).

Particularly preferred antisense probes comprise a nucleotide sequence selected from the group consisting of:

(vii) a nucleotide sequence that is complementary to a sequence having at least 80% identity to SEQ ID NO: 1;

(viii) a nucleotide sequence that is complementary to nucleotide residues 131-1400 of SEQ ID NO: 1 or a sequence having 80% identity thereto;

(ix) a nucleotide sequence that is complementary to nucleotide residues 1423-2239 of SEQ ID NO: 1 or a sequence having 80% identity thereto;

(x) a nucleotide sequence that is complementary to nucleotide residues 2264-2282 of SEQ ID NO: 1 or a sequence having 80% identity thereto;

(xi) a nucleotide sequence comprising at least 20 contiguous nucleotides of any one of (i) through (iv); and

(xii) an analog of any one of (i) through (v) as herein before defined.

Particularly preferred antisense nucleic acid probes in accordance with this embodiment of the invention comprise a nucleotide sequence selected from SEQ ID NO: 14, SEQ ID NO: 15, SEQ ID NO: 16, SEQ ID NO: 17, SEQ ID NO: 18, and SEQ ID NO: 19.

In another embodiment of the invention, the nucleic acid is an antagonist of GPR56 expression, such as, for example, an antisense nucleic acid, peptide nucleic acid (PNA), ribozyme, or interfering RNA, which is complementary, in whole or in part, to a target molecule comprising a sense strand, and can hybridize with the target molecule, in particular, GPR56-encoding RNA. When introduced into a cell using suitable methods, such a nucleic acid inhibits the expression of the GPR56 gene encoded by the sense strand. Antisense nucleic acid, ribozymes (eg. Cech et al., U.S. Ser. No. 4,987,071; Cech et al., U.S. Ser. No. 5,116,742; Bartel and Szostak, Science 261, 1411-1418, 1993), nucleic acid capable of forming a triple helix (eg. Helene, Anticancer Drug Res. 6, 569-584, 1991), PNAs (Hyrup et al., Bioorganic & Med. Chem. 4, 5-23, 1996; O'Keefe et al., Proc. Natl Acad. Sci. USA 93, 14670-14675, 1996), or interfering RNAs may be produced by standard techniques known to the skilled artisan, based upon the sequences disclosed herein.

Preferably, the antisense nucleic acid, ribozyme, PNA, or interfering RNA, comprises a sequence that is complementary to at least about 20 contiguous nucleotides of a sequence having at least about 80% identity to SEQ ID NO: 1 (ie. It is complementary to GPR56 RNA) and can hybridize thereto. For example, such antagonistic nucleic acid can be complementary to a target nucleic acid having the sequence of SEQ ID NO: 1 or a portion thereof sufficient to allow hybridization. Longer molecules, comprising a sequence that is complementary to at least about 25, or 30, or 35, or 40, or 45, or 50 contiguous nucleotides of GPR56 RNA are also encompassed by the present invention.

Antisense nucleic acids, ribozymes, PNAs, or interfering RNAs, are useful for a variety of purposes, including research and therapeutic applications.

For example, a construct comprising an antisense nucleic acid, ribozyme, PNA, or interfering RNA, can be introduced into a suitable cell to inhibit GPR56 expression and/or activity therein. Such a cell provides a valuable control cell, for instance in assessing the specificity of the GPR56 receptor-ligand interaction with the parent cell or other related cell types. In another embodiment, such a construct can be introduced into some or all of the cells of a mammal. The antisense nucleic acid, ribozyme, PNA, or interfering RNA, inhibits receptor expression, and any cancer, hyperproliferative or inflammatory process mediated by GPR56 receptors in the cells containing the construct are inhibited. Thus, a cancer, hyperproliferative response, inflammatory process, or inflammatory disease or condition, can be treated using an antisense nucleic acid, ribozyme, PNA, or interfering RNA, of the present invention.

Antibodies that can inhibit one or more functions characteristic of a GPR56 protein, such as a binding activity, a signalling activity, and/or stimulation of a cellular response, are also encompassed by the present invention. In one embodiment, antibodies of the present invention can inhibit binding of a ligand (i.e., one or more ligands) to a mammalian GPR56 protein and/or can inhibit one or more functions mediated by a mammalian GPR56 protein in response to ligand binding. In a particularly preferred embodiment, the antibodies can inhibit (reduce or prevent) the interaction of receptor with a natural ligand.

Accordingly, a fourth aspect of the invention provides a probe comprising an antibody that binds to a GPR56 polypeptide of the invention.

Preferred antibodies will selectively bind to a GPR56 polypeptide or an immunological derivative thereof and will not bind, or will only bind weakly, to non-GPR56 polypeptides or peptides.

Anti-GPR56 antibodies that are particularly contemplated by the present invention include monoclonal and polyclonal antibodies as well as fragments thereof comprising the antigen-binding domain and/or one or more complementarity determining regions of the native antibody. As used herein, the term “antibody fragment” shall be taken to mean a portion of the variable region of the immunoglobulin molecule that binds to its target, i.e., the antigen-binding region.

For some applications, it may be desirable to generate antibodies that specifically react with a particular human GPR56 isoform, in which case an epitope comprising amino acid residues 430-435 may be used as the antigen of choice to elicit antibody production.

Preferred antibodies for use in diagnostic imaging are those which react with an epitope in an extracellular region of the GPR56 polypeptide as expressed in a cancer cell. Such antibodies may be generated by using the complete human GPR56 polypeptide (SEQ ID NO: 2) as an immunogen. Alternatively, a peptide fragment derived from a predicted extracellular domain thereof, can be used. In this regard, the region of the of the polypeptide that is N-terminal to the first transmembrane domain, or a peptide fragment thereof, may be selected and screened for its ability to elicit the production of extracellular-specific anti-GPR56 antibodies using standard immunoassays, such as, for example, ELISA.

The anti-GPR56 antibodies of the invention may be particularly useful in diagnostic and prognostic assays for cancer, particularly the early or later detection of ovarian cancer or a metastasis thereof (eg. metastases in the omentum), by standard immunoassay or imaging methodologies. Similarly, such antibodies may be useful diagnosis and/or prognosis of any cancer in which GPR56 is expressed at a level that differs from normal or healthy tissue, such as, for example, prostate cancer.

Conventional methods can be used to prepare the antibodies. For example, by using an isolated GPR56 polypeptide or immunologically active derivative thereof, polyclonal antisera or monoclonal antibodies can be made using standard methods. For example, a mammal, (e.g., a mouse, hamster, or rabbit) can be immunized with the polypeptide or peptide to elicit an antibody response in the mammal. Techniques for conferring immunogenicity on a polypeptide include conjugation to carriers, or other techniques well known in the art. For example, the polypeptide can be administered in the presence of adjuvant or can be coupled to a carrier molecule known in the art, that enhances the immunogenicity of the polypeptide. The progress of immunization can be monitored by detection of antibody titres in plasma or serum. Standard ELISA or other immunoassay can be used to assess the titer of antibodies produced. Following immunization, antisera are obtained and, for example, IgG molecules corresponding to the polyclonal antibodies can be isolated from the antisera.

To produce monoclonal antibodies, antibody producing cells (lymphocytes) can be harvested from an animal immunized with the polypeptide or peptide, and fused with myeloma cells by standard somatic cell fusion procedures, thus immortalizing those cells and yielding hybridoma cells. Such techniques are well known in the art, for example, the hybridoma technique originally developed by Kohler and Milstein Nature 256: 495-499, 1975, as well as other techniques such as the human B-cell hybridoma technique (Kozbor et al., Immunol. Today 4: 72, 1983), the EBV-hybridoma technique to produce human monoclonal antibodies (Cole et al., In: Monoclonal antibodies in cancer therapy, Alan R. Bliss Inc., pp 77-96, 1985), and screening of combinatorial antibody libraries (Huse et al., Science 246, 1275-1281, 1989). Hybridoma cells are isolated and screened immunochemically for production of antibodies that are specifically reactive with the polypeptide and monoclonal antibodies isolated therefrom.

As with all immunogenic compositions for eliciting antibodies, the immunogenically effective amounts of the peptides of the invention must be determined empirically. Factors to be considered include the immunogenicity of the native polypeptide, whether or not the polypeptide will be complexed with or covalently attached to an adjuvant or carrier protein or other carrier, the route of administration for the composition, i.e., intravenous, intramuscular, subcutaneous, etc., and the number of immunizing doses to be administered. Such factors are known in the vaccine art and it is well within the skill of immunologists to make such determinations without undue experimentation.

The term “antibody” as used herein, is intended to include fragments thereof which are also specifically reactive with a polypeptide that mimics or cross-reacts with a B-cell epitope of a GPR56 polypeptide. Antibodies can be fragmented using conventional techniques and the fragments screened for utility in the same manner as described above for whole antibodies. For example, F(ab′)₂ fragments can be generated by treating antibody with pepsin. The resulting F(ab′)₂ fragment can be treated to reduce disulfide bridges to produce Fab′ fragments.

It is within the scope of this invention to include any secondary antibodies (monoclonal, polyclonal or fragments of antibodies), including anti-idiotypic antibodies, directed to the first mentioned antibodies discussed above. Both the first and second antibodies can be used in detection assays or a first antibody can be used with a commercially available anti-immunoglobulin antibody.

A further aspect of the present invention provides methods for detecting a cancer cell in a subject, said method comprising determining the level of GPR56-encoding nucleic acid or a GPR56 polypeptide in a sample of said subject.

As exemplified herein, GPR56-encoding mRNA is abundant in several cancer cell types, such as, for example, selected from the group consisting of prostate, omentum, ovary, liver, placenta, and brain, as determined by the appearance of RNA encoding said polypeptide in those tissues. Additionally, a positive correlation has been found between the over-expression of GPR56-encoding RNA in ovaries or omentum of ovarian cancer patients, and the occurrence of ovarian cancer and metastases thereof. Accordingly, the diagnostic methods described herein for the detection of GPR56-encoding nucleic acid or GPR56 polypeptides or a derivative thereof in a sample are particularly useful for the diagnosis or prognosis of ovarian cancer or a metastasis thereof.

The present inventors have also found that GPR56 is expressed only in a subset of memory T cells having immediate effector function. Such T cells lack the CCR7 receptor and are characterized further by rapid production of IFN-γ, IL-4, IL-5, or, in the case of CD8⁺ memory T cells, perforin granules. Additionally, effector memory T cells are characterized by their expression of tissue-specific homing receptors and receptors for inflammatory cytokines. The combination of rapid effector function and receptors that promote migration to peripheral sites suggests that CCR7⁻ memory T cells play a role in immediate responses to re-infection (Tussey et al., Eur. J. Immunol. 30, 1823-1829, 2000). The determination of GPR56 as a marker for such effector memory T cells indicates that GPR56 probes can be used to assist in the isolation of effector memory T cells and/or to determine the strength of an immune response of an individual to a particular antigenic determinant. For example, enhanced T cell-specific GPR56 expression in the serum of an individual, or at a particular site of inflammation, is indicative of enhanced effector memory T cells in said serum or at said inflammation site.

It will be apparent from the preceding discussion that many of the diagnostic methods provided by the present invention involve a degree of quantification to determine, on the one hand, the over-expression of GPR56 in tissue that is suspected of comprising a cancer cell, or, on the other hand, an enhanced number of effector memory T cells. Such quantification can be readily provided by the inclusion of appropriate control samples in the assays described below, derived from healthy or normal individuals. Alternatively, if internal controls are not included in each assay conducted, the control may be derived from an established data set that has been generated from healthy or normal individuals.

In the present context, the term “healthy individual” shall be taken to mean an individual who is known not to suffer from cancer, such knowledge being derived from clinical data on the individual, including, but not limited to, a different cancer assay to that described herein. As the present invention is particularly useful for the early detection of cancer, it is preferred that the healthy individual is asymptomatic with respect to the early symptoms associated with a particular cancer. In the case of ovarian cancer, early detection using well-known procedures is difficult, however reduced urinary frequency, rectal pressure, and abdominal bloating and swelling, are associated with the disease in its early stages, and, as a consequence, healthy individuals should not have any of these symptoms. Clearly, subjects suffering from later symptoms associated with ovarian cancer, such as, for example, metastases in the omentum, abdominal fluid, lymph nodes, lung, liver, brain, or bone, and subjects suffering from spinal cord compression, elevated calcium level, chronic pain, or pleural effusion, should also be avoided from the “healthy individual” data set.

The term “normal individual” shall be taken to mean an individual having a normal level of GPR56 expression in a particular sample derived from said individual. As will be known to those skilled in the art, data obtained from a sufficiently large sample of the population will normalize, allowing the generation of a data set for determining the average level of a particular parameter. Accordingly, the level of expression of GPR56 can be determined for any population of individuals, and for any sample derived from said individual, for subsequent comparison to GPR56 levels determined for a sample being assayed. Where such normalized data sets are relied upon, internal controls are preferably included in each assay conducted to control for variation.

It will also be apparent the detection of an effector memory T cell per se requires no such internal or external control, because GPR56 expression is restricted to a T cell sub-population comprising effector memory T cells.

In one embodiment, the present invention provides a method for detecting a cancer cell in a subject, said method comprising:

(i) determining the level of GPR56 mRNA expressed in a test sample from said subject; and

(ii) comparing the level of GPR56 mRNA determined at (i) to the level of GPR56 mRNA expressed in a comparable sample from a healthy or normal individual,

wherein a level of GPR56 mRNA at (i) that is enhanced in the test sample relative to the comparable sample from the normal or healthy individual is indicative of the presence of a cancer cell in said subject.

By “GPR56 mRNA” is meant mRNA encoding a GPR56 polypeptide that has at least about 80% identity to SEQ ID NO: 2, and, more particularly, mRNA comprising a nucleotide sequence that has at least about 80% identity, more preferably at least about 95% identity, and still more preferably at least about 99% identity to the nucleotide sequence set forth in SEQ ID NO: 1.

As discussed in detail below, the status of GPR56 mRNA in patient samples may be analyzed by a variety protocols that are well known in the art including in situ hybridization, northern blotting techniques, RT-PCR analysis (such as, for example, performed on laser capture microdissected samples), and microarray technology, such as, for example, using tissue microarrays probed with nucleic acid probes, or nucleic acid microarrays (ie. RNA microarrays or amplified DNA microarrays) microarrays probed with nucleic acid probes. All such assay formats are encompassed by the present invention.

For high throughput screening of large numbers of samples, such as, for example, public health screening of subjects, particularly human subjects, having a higher risk of developing cancer, microarray technology is a preferred assay format.

In a preferred embodiment, the level of GPR56 mRNA in the test sample is determined by hybridizing a GPR56 probe to GPR56-encoding RNA in the test sample under at least low stringency hybridization conditions and detecting the hybridization using a detection means.

Similarly, the level of GPR56 mRNA in the comparable sample from the healthy or normal individual is preferably determined by hybridizing a GPR56 probe to GPR56-encoding RNA in said comparable sample under at least low stringency hybridization conditions and detecting the hybridization using a detection means.

Preferably, the sample comprises ovarian tissue, prostate tissue, kidney tissue, uterine tissue, placenta, a cervical specimen, omentum, rectal tissue, brain tissue, bone tissue, lung tissue, lymphatic tissue, urine, semen, blood, abdominal fluid, or serum, or a cell preparation or nucleic acid preparation derived therefrom. More preferably, the sample comprises serum or abdominal fluid, or a tissue selected from the group consisting of: ovary, lymph, lung, liver, brain, placenta, brain, omentum, and prostate. Even more preferably, the sample comprises serum or abdominal fluid, ovary (eg. OSE), or lymph node tissue. The sample can be prepared on a solid matrix for histological analyses, or alternatively, in a suitable solution such as, for example, an extraction buffer or suspension buffer, and the present invention clearly extends to the testing of biological solutions thus prepared.

The GPR56 probe may be any nucleic acid probe described herein above. As will be known to those skilled in the art, shorter probes are hybridized at lower stringency hybridization (ie. reduced temperature and/or higher salt concentration and/or higher detergent concentration) than longer nucleic acid probes. Generally, hybridization is carried out well below the calculated melting temperature (Tm) of a DNA duplex comprising the probe. Riboprobes are particularly preferred for applications utilizing oligonucleotides as RNA/RNA duplexes are more stable. For example, the oligonucleotide probes exemplified herein have calculated Tm values in the range of about 55° C. to about 60° C., suggesting that hybridization involving such probes should be carried out at a temperature in the range of ambient temperature to about 45° C., and more preferably between about 40° C. to about 45° C. (ie. low stringency to moderate stringency conditions). This contrasts with standard hybridization temperatures of about 65° C. for nucleic acid probes of about 100 nucleotides or longer (ie. moderate to high stringency hybridization conditions).

For the purposes of defining the level of stringency to be used in these diagnostic assays, a low stringency is defined herein as being a hybridization and/or a wash carried out in 6×SSC buffer, 0.1% (wlv) SDS at 28° C., or equivalent conditions. A moderate stringency is defined herein as being a hybridization and/or washing carried out in 2×SSC buffer, 0.1% (w/v) SDS at a temperature in the range 45° C. to 65° C., or equivalent conditions. A high stringency is defined herein as being a hybridization and/or wash carried out in 0.1×SSC buffer, 0.1% (w/v) SDS, or lower salt concentration, and at a temperature of at least 65° C., or equivalent conditions. Reference herein to a particular level of stringency encompasses equivalent conditions using wash/hybridization solutions other than SSC known to those skilled in the art.

Generally, the stringency is increased by reducing the concentration of SSC buffer, and/or increasing the concentration of SDS and/or increasing the temperature of the hybridization and/or wash. Those skilled in the art will be aware that the conditions for hybridization and/or wash may vary depending upon the nature of the hybridization matrix used to support the sample RNA, or the type of hybridization probe used.

In general, the sample or the probe is immobilized on a solid matrix or surface (e.g., nitrocellulose). For high throughput screening, the sample or probe will generally comprise an array of nucleic acids on glass or other solid matrix, such as, for example, as described in WO 96/17958. Techniques for producing high density arrays are described, for example, by Fodor et al., Science 767-773, 1991, and in U.S. Pat. No. 5,143,854. Typical protocols for other assay formats can be found, for example in Current Protocols In Molecular Biology, Unit 2 (Northern Blotting), Unit 4 (Southern Blotting), and Unit 18 (PCR Analysis), Frederick M. Ausubul et al. (ed)., 1995.

The detection means according to this aspect of the invention may be any nucleic acid-based detection means such as, for example, nucleic acid hybridization or amplification reaction (eg. PCR), a nucleic acid sequence-based amplification (NASBA) system, inverse polymerase chain reaction (iPCR), in situ polymerase chain reaction, or reverse transcription polymerase chain reaction (RT-PCR), amongst others.

The probe can be labelled with a reporter molecule capable of producing an identifiable signal (e.g., a radioisotope such as ³²P or ³⁵S, or a fluorescent or biotinylated molecule). According to this embodiment, those skilled in the art will be aware that the detection of said reporter molecule provides for identification of the probe and that, following the hybridization reaction, the detection of the corresponding nucleotide sequences in the sample is facilitated. Additional probes can be used to confirm the assay results obtained using a single probe.

Wherein the detection means is an amplification reaction such as, for example, a polymerase chain reaction or a nucleic acid sequence-based amplification (NASBA) system or a variant thereof, one or more nucleic acid probes molecules of at least about 20 contiguous nucleotides in length is hybridized to mRNA encoding GPR56, or alternatively, hybridized to cDNA or cRNA produced from said mRNA, and nucleic acid copies of the template are enzymically-amplified.

Those skilled in the art will be aware that there must be a sufficiently high percentage of nucleotide sequence identity between the probes and the RNA sequences in the sample template molecule for hybridization to occur. As stated previously, the stringency conditions can be selected to promote hybridization.

In one format, PCR provides for the hybridization of non-complementary probes to different strands of a double-stranded nucleic acid template molecule (ie. a DNA/RNA, RNA/RNA or DNA/DNA template), such that the hybridized probes are positioned to facilitate the 5′-to 3′ synthesis of nucleic acid in the intervening region, under the control of a thermostable DNA polymerase enzyme. In accordance with this embodiment, one sense probe and one antisense probe as described herein would be used to amplify DNA from the hybrid RNA/DNA template or cDNA.

In the present context, the cDNA would generally be produced by reverse transcription of mRNA present in the sample being tested (ie. RT-PCR). RT-PCR is particularly useful when it is desirable to determine expression of a GPR56-encoding gene. It is also known to those skilled in the art to use mRNA/DNA hybrid molecules as a template for such amplification reactions, and, as a consequence, first strand cDNA synthesis is all that is required to be performed prior to the amplification reaction.

Variations of the embodiments described herein are described in detail by McPherson et al., PCR: A Practical Approach. (series eds, D. Rickwood and B. D. Hames), IRL Press Limited, Oxford. pp 1-253, 1991.

The amplification reaction detection means described supra can be further coupled to a classical hybridization reaction detection means to further enhance sensitivity and specificity of the inventive method, such as by hybridizing the amplified DNA with a probe which is different from any of the probes used in the amplification reaction.

Similarly, the hybridization reaction detection means described supra can be further coupled to a second hybridization step employing a probe which is different from the probe used in the first hybridization reaction.

The comparison to be performed in accordance with the present invention may be a visual comparison of the signal generated by the probe, or alternatively, a comparison of data integrated from the signal, such as, for example, data that have been corrected or normalized to allow for variation between samples. Such comparisons can be readily performed by those skilled in the art.

In an alternative embodiment, the present invention provides a method for determining an effector memory T cell response in a subject, said method comprising:

(i) determining the level of GPR56 mRNA expressed in a test sample from said subject; and

(ii) comparing the level of GPR56 mRNA determined at (i) to the level of GPR56 mRNA expressed in a comparable sample from a healthy or normal individual,

wherein a level of GPR56 mRNA at (i) that is enhanced in the test sample relative to the comparable sample from the normal or healthy individual is indicative of an effector memory T cell response in the subject.

In an alternative embodiment, the present invention provides a method for determining whether or not a subject has been re-infected with an infectious agent, said method comprising:

(i) determining the level of GPR56 mRNA expressed in a test sample from said subject; and

(ii) comparing the level of GPR56 mRNA determined at (i) to the level of GPR56 mRNA expressed in a comparable sample from a healthy or normal individual,

wherein a level of GPR56 mRNA at (i) that is enhanced in the test sample relative to the comparable sample from the normal or healthy individual is indicative of re-infection in the subject.

This embodiment of the invention is readily performed using the assay formats described supra. Preferably, the test sample is blood or whole serum or a fraction thereof comprising T cells, such as, for example, buffy coat. Other tissues, such as those described supra are not excluded.

In an alternative embodiment, the present invention provides a method for determining the presence of effector memory T cells in a test sample, said method comprising:

(i) hybridizing a GPR56 probe to GPR56-encoding RNA in the test sample under at least low stringency hybridization conditions; and

(ii) detecting the hybridization using a detection means,

wherein said hybridization is indicative of the presence of an effector memory T cell in said test sample.

This embodiment of the invention is readily performed using the assay formats described supra, however no external or internal control, and no comparison is required, to perform this embodiment of the invention.

A further embodiment of the invention provides a process for counting effector memory T cells in a subject comprising for determining the presence of effector memory T cells in a test sample as described herein and normalizing the hybridization signal to determine T cell count.

The status of GPR56 gene expression in a subject may be also be determined at the protein level, using the peptides and antibodies described herein above, in combination with a variety protocols that are well known in the art, including immunohistochemical analysis, western blot analysis, ELISA or other immunoassay, and microarray technology, such as, for example, using tissue microarrays probed with antibodies. As with nucleic acid screens, high throughput screening is preferred for large numbers of samples.

Accordingly, a further embodiment of the invention provides a method for detecting a cancer cell in a subject, said method comprising:

(i) determining the level of a GPR56 polypeptide in a test sample from said subject; and

(ii) comparing the level of GPR56 polypeptide determined at (i) to the level of said GPR56 polypeptide in a comparable sample from a healthy or normal individual,

wherein a level of said GPR56 polypeptide at (i) that is enhanced in the test sample relative to the comparable sample from the normal or healthy individual is indicative of the presence of a cancer cell in said subject.

By “GPR56 polypeptide” is meant a polypeptide that comprises an amino acid sequence having at least about 80% identity to SEQ ID NO: 2, more preferably at least about 95% identity, and still more preferably at least about 99% identity to the sequence set forth in SEQ ID NO: 2.

Preferably, the subject is human. Samples from the subject will be those samples that are suitable for screening using nucleic acid probes, however histological specimens are particularly amenable to antibody-based detection.

In an alternative embodiment, the present invention provides a method for determining whether or not a subject has been re-infected with an infectious agent, said method comprising:

(iii) determining the level of a GPR56 polypeptide in a test sample from said subject; and

(iv) comparing the level of the GPR56 polypeptide determined at (i) to the level of said GPR56 polypeptide in a comparable sample from a healthy or normal individual,

wherein a level of said GPR56 polypeptide at (i) that is enhanced in the test sample relative to the comparable sample from the normal or healthy individual is indicative of re-infection in the subject.

Preferably, the test sample used in performing this embodiment of the invention is blood or whole serum or a fraction thereof comprising T cells, such as, for example, buffy coat. Other tissues, such as those described supra are not excluded.

Preferably, the level of GPR56 polypeptide in the test sample is determined by a process comprising:

(i) contacting said sample with an antibody that binds to a GPR56 polypeptide under conditions sufficient for binding to occur; and

(ii) determining the binding.

Similarly, the level of GPR56 mRNA in the comparable sample from the healthy or normal individual is preferably determined by a process comprising:

(i) contacting said sample with an antibody that binds to a GPR56 polypeptide under conditions sufficient for binding to occur; and

(ii) determining the binding.

Standard assays are used to determine binding of the antibody to the GPR56 polypeptide in the samples, such as, for example, ELISA, radioimmunoassay, western blot immunoassay, amongst others. Protocols are provided, for example, by Ausubel et al (supra).

In an alternative embodiment, the present invention provides a method for determining the presence of effector memory T cells in a test sample, said method comprising:

(i) contacting said sample with an antibody that binds to a GPR56 polypeptide under conditions sufficient for binding to occur; and

(ii) determining the binding.

wherein binding of the antibody to the test sample is indicative of the presence of an effector memory T cell in said test sample.

A further embodiment of the invention provides a process for counting effector memory T cells in a subject comprising for determining the presence of effector memory T cells in a test sample as described herein and normalizing the antibody binding signal, such as, for example, a signal in an ELISA assay or radio immunoassay that is provided by the reporter (ie. second antibody conjugated to horseradish peroxidase, alkaline phosphatase, or I¹²⁵-label, amongst others) to determine T cell count.

Modulation of GPR56 function according to the present invention, through the inhibition or promotion of at least one function characteristic of a mammalian GPR56 protein, provides an effective and selective way of inhibiting or promoting receptor-mediated functions. As GPR56 is selectively expressed on effector memory T cells, mammalian GPR56 proteins provide a target for selectively interfering with or promoting effector memory T cell function in a mammal, such as a human. Once lymphocytes are recruited to a site, other leukocyte types, such as monocytes, may be recruited by secondary signals. Thus, agents which inhibit or promote GPR56 function, including ligands, inhibitors and/or promoters, such as those identified as described herein, can be used to modulate leukocyte function (e.g., leukocyte infiltration including recruitment and/or accumulation), particularly of lymphocytes, for therapeutic purposes.

In one aspect, the present invention provides a method of inhibiting or promoting an inflammatory response in an individual in need of such therapy, comprising administering an agent which inhibits or promotes mammalian GPR56 function to an individual in need of such therapy. In one embodiment, a compound which inhibits one or more functions of a mammalian GPR56 protein (e.g., a human GPR56) is administered to inhibit (i.e., reduce or prevent) inflammation. For example, anti-GPR56 antibodies of the present invention, or antagonistic nucleic acid (antisense nucleic acid, PNA, interfering RNA, ribozyme, etc) can be used in the method. As a result, one or more hyperproliferative disorders, inflammatory processes, such as leukocyte emigration, chemotaxis, exocytosis (e.g., of enzymes) or inflammatory mediator release, is inhibited. For example, leukocytic infiltration of inflammatory sites (e.g., in a delayed-type hypersensitivity response) can be inhibited according to the present method.

In a further embodiment, a compound that inhibits one or more functions of a mammalian GPR56 protein (e.g., a human GPR56) is administered to prevent, inhibit, or delay tumor growth, particularly in the treatment of carcinoma, such as, for example, an epitehlial carcinoma, and more particularly, in the case of ovarian cancer or a metastasis thereof. For example, anti-GPR56 antibodies of the present invention, or antagonistic nucleic acid (antisense nucleic acid, PNA, interfering RNA, ribozyme, etc) can be used in the method.

In another embodiment, an agent (e.g., receptor agonist) which promotes one or more functions of a mammalian GPR56 protein (e.g., a human GPR56) is administered to induce (trigger or enhance) an inflammatory response, such as leukocyte emigration, chemotaxis, exocytosis (e.g., of enzymes) or inflammatory mediator release, resulting in the beneficial stimulation of inflammatory processes. For example, natural killer cells can be recruited to combat viral infections or neoplastic disease.

The term “individual” is defined herein to include animals such as mammals, including, but not limited to, primates (e.g., humans), cows, sheep, goats, horses, dogs, cats, rabbits, guinea pigs, rats, mice or other bovine, ovine, equine, canine, feline, rodent or murine species. Diseases and conditions associated with inflammation, infection, and cancer can be treated using the method. In a preferred embodiment, the disease or condition is one in which the actions of lymphocytes, particularly effector memory T cells, are to be inhibited or promoted for therapeutic (including prophylactic) purposes. In a particularly preferred embodiment, the inflammatory disease or condition is a T cell-mediated disease or condition.

Diseases or conditions, including chronic diseases, of humans or other species which can be treated with inhibitors of GPR56 function, include, but are not limited to: inflammatory or allergic diseases and conditions, including systemic anaphylaxis or hypersensitivity responses, drug allergies (e.g., to penicillin, cephalosporins), insect sting allergies; inflammatory bowel diseases, such as Crohn's disease, ulcerative colitis, ileitis and enteritis; vaginitis; psoriasis and inflammatory dermatoses such as dermatitis, eczema, atopic dermatitis, allergic contact dermatitis, urticaria; vasculitis (e.g., necrotizing, cutaneous, and hypersensitivity vasculitis); spondyloarthropathies; scleroderma; respiratory allergic diseases such as asthma, allergic rhinitis, hypersensitivity lung diseases, hypersensitivity pneumonitis, interstitial lung diseases (ILD) (e.g., idiopathic pulmonary fibrosis, or ILD associated with rheumatoid arthritis, or other autoimmune conditions); autoimmune diseases, such as arthritis (e.g., rheumatoid arthritis, psoriatic arthritis), multiple sclerosis, systemic lupus erythematosus, myasthenia gravis, diabetes, including diabetes mellitus and juvenile onset diabetes, glomerulonephritis and other nephritides, autoimmune thyroiditis, Behcet's disease; graft rejection (e.g., in transplantation), including allograft rejection or graft-versus-host disease; other diseases or conditions in which undesirable inflammatory responses are to be inhibited can be treated, including, but not limited to, atherosclerosis, cytokine-induced toxicity, myositis (including polymyositis, dermatomyositis).

Diseases or conditions of humans or other species which can be treated with agonists of GPR56 function, include, but are not limited to: diseases in which angiogenesis or neovascularization plays a role, including neoplastic disease, retinopathy (e.g., diabetic retinopathy), and macular degeneration; infectious diseases, such as bacterial infections and tuberculoid leprosy, and especially viral infections; immunosuppression, such as that in individuals with immunodeficiency syndromes such as AIDS, individuals undergoing radiation therapy, chemotherapy, or other therapy which causes immunosuppression; immunosuppression due congenital deficiency in receptor function or other causes; and re-infection. Agonists of GPR56 function can also have protective effects useful to combat stem cell depletion during cancer chemotherapy (Sarris, A. H. et al., J. Exp. Med., 178, 1127-1132, 1993).

According to the method, one or more agents can be administered to the host by an appropriate route, either alone or in combination with another drug. An effective amount of a nucleic acid or antibody agent having antagonist or agonist activity is administered. An effective amount is an amount sufficient to achieve the desired therapeutic or prophylactic effect, under the conditions of administration, such as an amount sufficient for inhibition or promotion of GPR56 receptor function, and thereby, inhibition or promotion, respectively, of a receptor-mediated process (e.g., an inflammatory response).

A variety of routes of administration are possible including, but not necessarily limited to oral, dietary, topical, parenteral (e.g., intravenous, intraarterial, intramuscular, subcutaneous injection), and inhalation (e.g., intrabronchial, intranasal or oral inhalation, intranasal drops) routes of administration, depending on the agent and disease or condition to be treated. For respiratory allergic diseases such as asthma, inhalation is a preferred mode of administration.

Formulation of an agent to be administered will vary according to the route of administration selected (e.g., solution, emulsion, capsule). An appropriate composition comprising the agent to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluid, nutrient or electrolyte replenishers and the like (See, generally, Remington's Pharmaceutical Sciences, 17th Edition, Mack Publishing Co., Pa., 1985). For inhalation, the agent can be solubilized and loaded into a suitable dispenser for administration (e.g., an atomizer, nebulizer or pressurized aerosol dispenser).

Furthermore, where the agent is a protein or peptide, the agent can be administered via in vivo expression of the recombinant protein. In vivo expression can be accomplished via somatic cell expression according to suitable methods (see, e.g. U.S. Pat. No. 5,399,346). In this embodiment, nucleic acid encoding the protein can be incorporated into a retroviral, adenoviral or other suitable vector (preferably, a replication deficient infectious vector) for delivery, or can be introduced into a transfected or transformed host cell capable of expressing the protein for delivery. In the latter embodiment, the cells can be implanted (alone or in a barrier device), injected or otherwise introduced in an amount effective to express the protein in a therapeutically effective amount.

The present invention is further described by the following non-limiting Examples.

### EXAMPLE 1

Isolation of a cDNA Encoding GPR56 and Characterization of Homologous GPR56-Encoding Sequences in Mammals

Degenerate oligonucleotides corresponding to conserved regions within the human Glucagon-like peptide receptor (GLP1) family were used to amplify specific DNA sequences from a variety of cDNA libraries. DNA from human hypothalamus and heart yielded fragments of the expected size for generating libraries. These fragments were subcloned and sequenced. One fragment, 456 bp long, revealed a novel 7 TM receptor sequence distantly related to the secretin receptor family.

A human heart cDNA library (Stratagene) was screened by standard hybridization using the fragment supra, under high stringency conditions. Two positive hybridizing clones were identified, of 1 and 2.9 kb in size, respectively. Nucleotide sequence analysis revealed that the longer clone contains an open reading frame of 2079 nucleotides encoding a 693 amino acid long protein (designated GPR56 or TSR32). The nucleotide and putative amino acid sequence of this clone is provided in SEQ ID NOS: 1 and 2, respectively.

A search of the EMBL/GenBank database revealed low but significant sequence similarities between SEQ ID NO: 2 and polypeptides of the secretin receptor family of 7 TM receptors. In particular, the percentage identity between SEQ ID NO: 2 and other 7 TM polypeptides was only about 25% to 35%, and homology was limited to the transmembrane region. The highest overall sequence similarity was found to four other G-protein coupled receptor subtypes, the human epididymis-specific HE6 (31% identity), the human lymphocyte antigen CD97 (33% identity), the orphan human receptor EMR1 (27% identity), the insect DHR (26% identity) and the rat latrophilin-related protein 1 precursor (35% identity) all of which share a large extracellular domain with numerous putative O- and N-glycosylation sites. Except for DHR, in all of the receptors there is a cysteine motif preceding the first transmembrane domain. Further, in contrast to the CD97 and EMR1 receptors, TSR32 does not contain any EGF-domains or calcium binding sites in the extracellular domain.

Interestingly, at the gene level the exon/intron organization of the TSR32-encoding cDNA, at least within the region encoding the transmembrane domain, is most similar to the gene structure of the PACAP receptor family, with fewer but identical exon/intron borders.

Subsequent BLAST searching revealed homologs of the originally-isolated GPR56-encoding cDNA clone (ie. homologs of SEQ ID NOS: 1 and 2). In particular, using the default parameters of the BLAST program at the NCBI database, three additional GPR56-encoding nucleotide sequences from humans or human cell lines were identified, having about 99% identity to SEQ ID NO: 1. The different human GPR56 alleles have been designated as GPR56-1, GPR56-2, GPR56-3, and GPR56-4, with GPR56-1 being the originally-isolated sequence. The nucleotide sequences of GPR56-2, GPR56-3, and GPR56-4 are set forth in SEQ ID NOS: 3, 5, and 7, respectively.

The GPR56 polypeptides encoded by these homologs, which are set forth in SEQ ID NOS: 4, 6, and 8, are also highly conserved with the amino acid sequence set forth in SEQ ID NO: 2, as evidenced by the alignment shown in FIG. 1. In particular, the amino acid sequences of GPR56-1 (SEQ ID NO: 2) and GPR56-3 (SEQ ID NO: 6) are 100% identical, and GPR56-2 (SEQ ID NO: 4) differs from this isoform only by the conservative substitution of Gln306 for His306. In contrast, the amino acid sequence of GPR56-4 (SEQ ID NO: 8) comprises a six amino acid deletion at positions 430-435 relative to the other two isoforms. These data indicate the existence of a small multigene family encoding GPR56 in humans.

Additionally, BLAST searching using the default parameters of the BLAST program at the NCBI database has revealed a murine homolog of human GPR56-1 that is about 83% identical thereto over its entire length. The nucleotide sequence of the murine GPR56-encoding gene is set forth in SEQ ID NO: 9 and the derived amino acid sequence is set forth in SEQ ID NO: 10. Alignment of the amino acid sequence of the murine and human genes (FIG. 1) reveals that the murine sequence also comprises the same amino acid deletion present in human GPR56-4, and is most highly identical to that isoform.

### EXAMPLE 2

Expression Analysis of the GPR56 Gene

Northern and expression microarray analyses indicates a low level of expression of human GPR56 mRNA in a large variety of tissues including thyroid, prostate, ovary, omentum, kidney, lung, cerebellum, and heart. The highest level of GPR56 mRNA was found in the thyroid gland. This expression pattern of the receptor mRNA indicates that GPR56 has important functions in metabolic regulations throughout the body, possibly mediated via the thyroid gland.

Additionally, the human GPR56 homologs, GPR56-2, and GPR56-4, were isolated as cDNAs from placental choriocarcinoma cells and brain anaplastic oligodendroglioma, indicating that GPR56 is also expressed in the placenta and brain.

Similarly, the murine GPR56 was isolated from hemopoietic cells of mice.

The gene encoding GPR56 has been mapped to human chromosome 16q31 using in situ hybridization. This localization has been confirmed by radiation hybrid analysis. An autosomal recessive disorder (Bardet Biedi Syndrome; Kwitek-Black, A. E. et al., Nature Genetics 5:392-396, 1993) has also been linked to this region. Features of this syndrome include obesity, retinal degeneration, hypogonadism and mental retardation. The expression of GPR56 examined by in situ hybridization and Northern analysis overlaps strongly with the tissues affected in the Bardet Biedl Syndrome making GPR56 a good candidate for this locus.

### EXAMPLE 3

Identification of Unique Nucleic Acid Probes for Detecting GPR56 mRNA

The nucleotide sequence set forth in SEQ ID NO: 1 was analyzed by the criteria described herein above to identify suitable probes for use in diagnostic applications relating to the expression or over-expression of GPR56 mRNA. In particular, analysis was limited to sequences comprising about 20-30 contiguous nucleotides of SEQ ID NO: 1.

In particular, SEQ ID NO: 1 was subjected to a BLAST search (Altschul et al., J. Mol. Biol. 215, 403-410, 1990), and the alignments that were generated were analyzed to identify regions within the entire sequence that were not present in genes other than human GPR56-encoding genes.

The nucleotide sequences of unique regions within SEQ ID NO: 1 were also analyzed for such 20-mer to 30-mer regions that satisfied the following criteria:

(i) it comprises less than ten(10) A residues;

(ii) it comprises less than ten(10) T residues;

(iii) it comprises less than nine(9) C residues;

(iv) it comprises less than nine(9) G residues;

(v) it comprises less than seven(7) A residues in any window consisting of 8 nucleotides;

(vi) it comprises less than seven(7) T residues in any window consisting of 8 nucleotides;

(vii) it comprises less than eight(8) C residues in any window consisting of 8 nucleotides;

(viii) it comprises less than eight(8) G residues in any window consisting of 8 nucleotides;

(ix) it comprises less than six(6) consecutive A residues;

(x) it comprises less than six(6) consecutive T residues;

(xi) it comprises less than five(5) consecutive C residues; and

(xii) it comprises less than five(5) consecutive G residues.

Furthermore, the self-complementarity of those unique sequences satisfying the above criteria was assessed, by aligning each sequence with its reverse complement.

Suitable nucleotide sequences were identified as being potentially useful probes, and these are set forth in Table 1 (SEQ ID NOS: 11-19).

### EXAMPLE 4

Restriction of GPR56 Gene Expression in T Cells to Effector Memory (ie. CCR7⁻) T Cells

Introduction

T cells are at the heart of the adaptive immune response. Our ability to combat pathogenic infections depends to a large extent on the ability of our immune system to remember past infections. This phenomenon is known as immunological memory. Although well recognized and the principal behind vaccinations we still understand the process of immunological memory very poorly. A typical primary immune response would involve activation and clonal expansion of antigen-specific T cells and differentiation into effector T cells. While most of these effector cells are short-lived and will die shortly after the antigen is cleared, a few antigen-experienced cells persist for a longer-time and are known as memory cells which confer long term protection. It is still unclear whether memory cells arise from fully differentiated effector cells or through a separate pathway.

Memory T cells can be divided into two broad categories on the basis of their is activation status and expression of chemokine receptor CCR7. CCR7 controls homing to secondary lymphoid organs. CCR7⁻ memory T cells (effector memory) which express receptors for migration to sites of inflammation and possess immediate effector functions. On the other hand, CCR7⁺ memory T cells (central memory) express receptors for homing to lymph nodes and are in a quiescent state lacking immediate effector functions. GPR56 gene expression was compared in the two memory subsets using Affymetrix microarray technology.

Experimental Methods

3. Cell Isolation

Peripheral blood was collected from a volunteer and peripheral blood mononuclear cells (PBMC) were isolated using Ficoll density centrigugation. These cells were then enriched using RosetteSep kit as per manufacturer's instructions. Enriched cells were labeled with fluorescent antibodies CD4 FITC (marker for T helper subset of T cells), CCR7 PE (lymphoid tissue homing receptor) and CD45RO APC (marker for memory T cells). Labeled cells were then sorted into two distinct populations using FACS Star cell sorter. Total RNA was isolated from cells using the Rneasy Total RNA Isolation kit (Qiagen) as per manufacturer's instructions.

4. Preparation of cRNA and GeneChip Hybridization

cDNA was specifically transcribed from the poly-A mRNA using a poly-T nucleotide primer, containing a T7 RNA polymerase promoter (GeneWorks, Australia). Biotinylated, antisense target cRNA was subsequently synthesized by in vitro transcription, using the Enzo BioArray High Yield RNA Transcript Labeling kit. The biotin-labeled target cRNA was then fragmented, and used to prepare a hybridization cocktail, which included probe array controls and blocking agents. This cocktail was initially hybridized to test arrays to evaluate the quality of the cRNA, and then to U95A arrays containing approximately 12,000 human genes for expression analysis. Washing and staining of the hybridized probe array were performed by an automated fluidics station, according to the manufacturer's protocols. The stained array was then scanned and the resultant image captured as a data image file.

5. Data Analysis

From data image files, gene transcript levels were determined using algorithms in the GeneChip Analysis Suit software (Affymetrix). The expression levels of all genes on the array set were compared between type 1 and type 2 cells, with differences of 2-fold or larger likely to reflect significant changes in gene expression. Genes that showed a change of 2-fold or greater in at least two separate experiments were considered as differentially expressed. Each probe was assigned a call of present (expressed) or absent (not expressed) using Affymetrix decision matrix.

6. Results

Results were analyzed using Affymetrix software. We identified a large number of genes differentially expressed between central and effector memory subsets of T cells. GPR56 was specifically expressed by effector memory cells whereas CCR7 was expressed on central memory T cells (data not provided).

### EXAMPLE 5

Association between GPR56 Expression and Ovarian Cancer or Metastases Thereof

GeneChips were prepared and hybridized to labeled single-strand probes prepared from ovary or omentum RNA, using standard procedures. The RNA samples were either from a number of healthy subjects, or from a number of subjects having ovarian cancer at various stages of the disease, including those subjects having early symptoms of ovarian cancer, or alternatively, metastases in the omentum. Samples from subjects having cancer of the prostate cancer were also analyzed (data not shown).

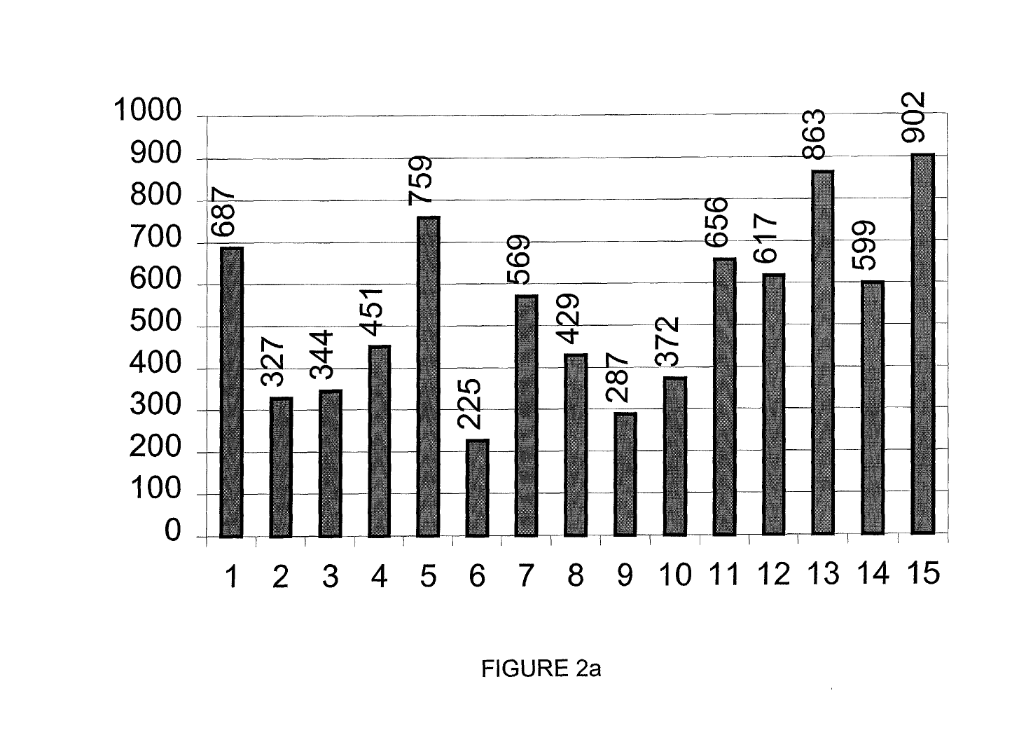

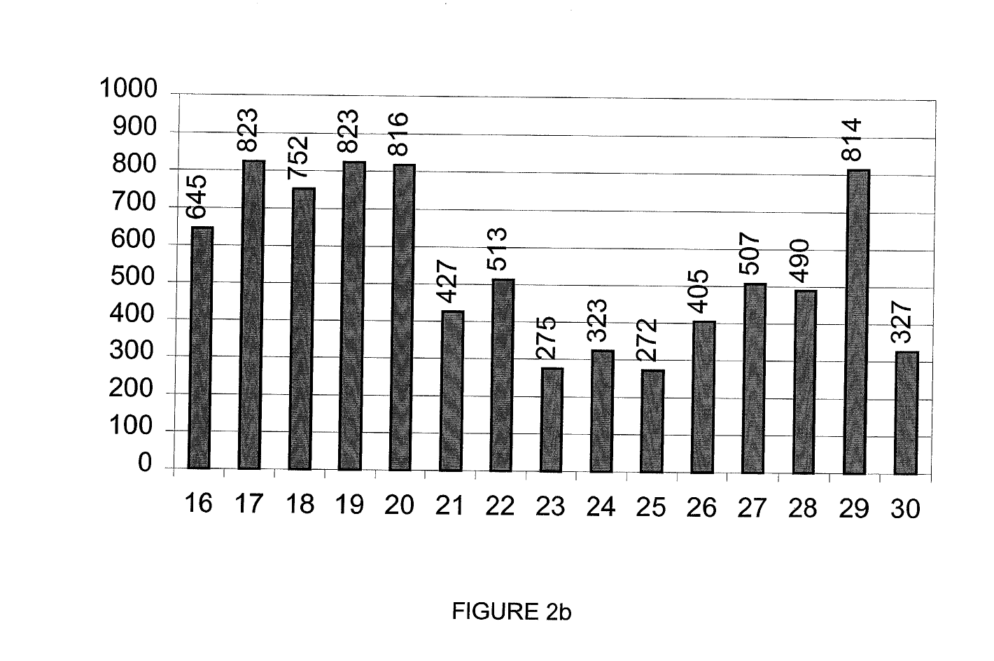

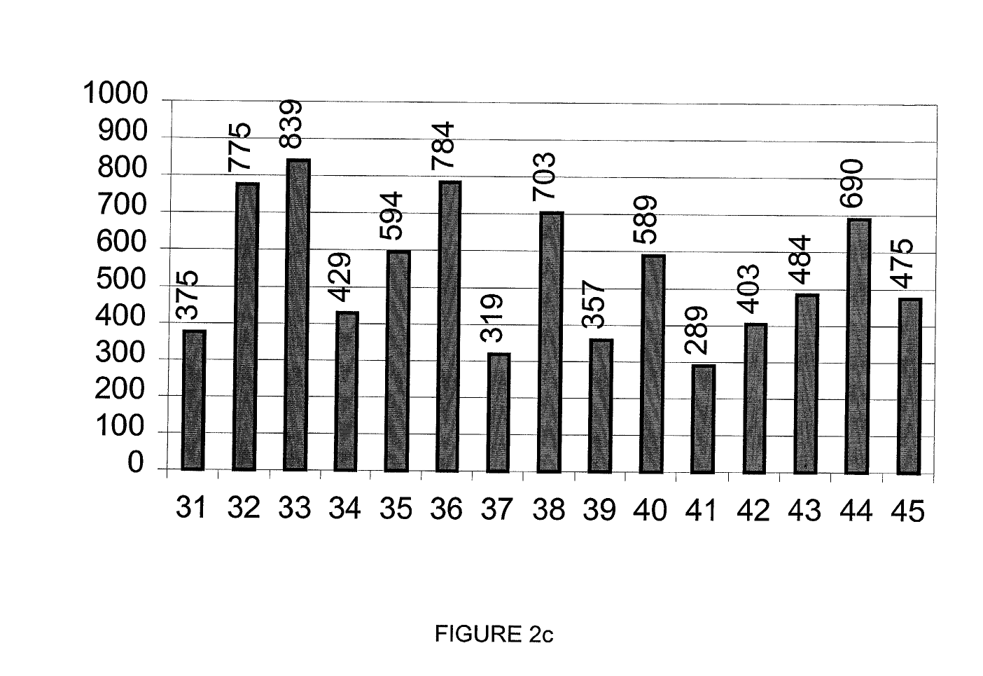

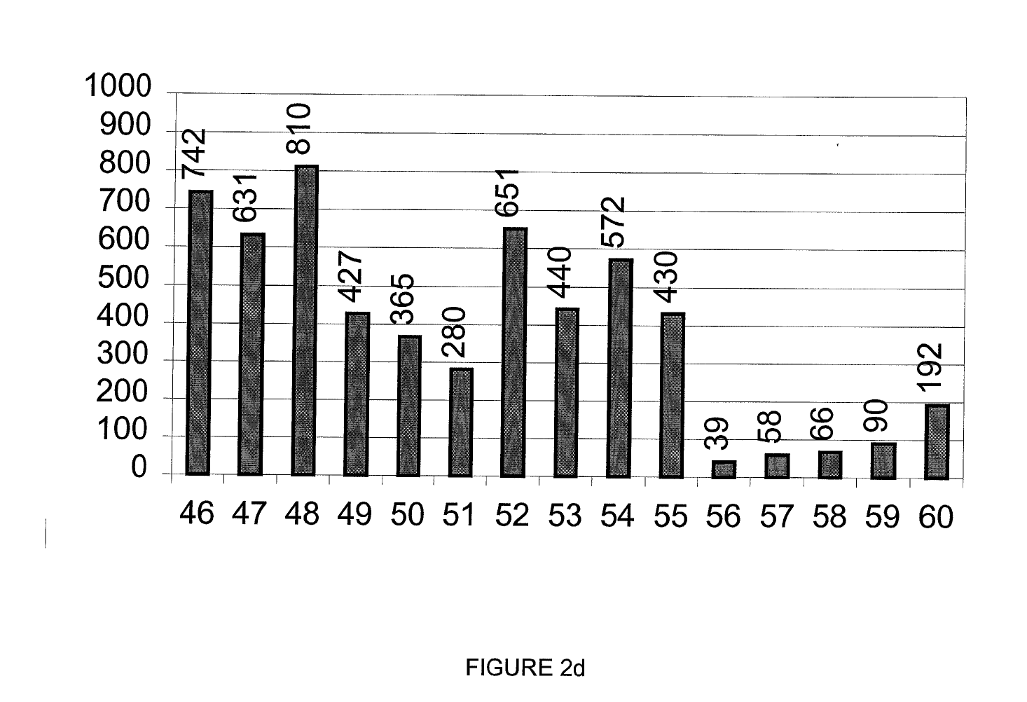

Data shown in FIG. 2 indicate that there is a significant enhancement of GPR56 expression detected in subjects having early stage or advanced ovarian cancer, relative to healthy subjects. The level of enhancement of GPR56 expression in subjects having cancer is at least about 6-fold.

High levels of expression are also seen in the omentum of ovarian cancer patients having secondary tumors of the omentum, relative to the level present in normal omentum tissue subjects (FIG. 2).

Similarly, high levels of expression were observed in subjects having prostate cancer (not shown).

We claim:

1. An isolated nucleic acid comprising a nucleotide sequence that encodes a GPR56 polypeptide or an immunologically active derivative thereof.

2. The isolated nucleic acid of claim 1, wherein said nucleic acid encodes a human GPR56 polypeptide.

3. The isolated nucleic acid of claim 1 comprising a nucleotide sequence that is at least 80% identical to SEQ ID NO: 1.

4. The isolated nucleic acid of claim 1 wherein said nucleic acid hybridizes under moderate or high stringency conditions to a sequence that is complementary to SEQ ID NO: 1.

5. The isolated nucleic acid of claim 3 wherein the percentage identity to SEQ ID NO: 1 is at least about 95%.

6. The isolated nucleic acid of claim 3 wherein the percentage identity to SEQ ID NO: 1 is about 99%.

7. The isolated nucleic acid of claim 4 wherein the hybridization comprises a hybridization or wash buffer comprising a parameter selected from the group consisting of:
(i) a salt concentration that is equivalent to 0.1×SSC-0.2×SSC buffer or lower salt concentration;
(ii) a detergent concentration equivalent to 0.1% (w/v) SDS or higher; and
(iii) an incubation temperature of about 45° C. to 65° C. or higher.

8. A gene construct comprising the isolated nucleic acid of claim 1 in operable connection with a promoter sequence.

9. An isolated nucleic acid comprising a nucleotide sequence selected from the group consisting of:
(i) the nucleotide sequence set forth in SEQ ID NO: 1;
(ii) nucleotide residues 163 to 2241 of SEQ ID NO: 1;
(iii) a nucleotide sequence that encodes the amino acid sequence set forth in SEQ ID NO: 2; and
(iv) a nucleotide sequence that is complementary to any one of (i) to (iii).

10. A gene construct comprising the isolated nucleic acid of claim 9 in operable connection with a promoter sequence.

11. An isolated GPR56 polypeptide or an immunologically active derivative thereof, substantially free of conspecific proteins.

12. The isolated GPR56 polypeptide of claim 11 comprising an amino acid sequence that is at least 80% identical to SEQ ID NO: 2.

13. The isolated GPR56 polypeptide of claim 12 wherein the percentage identity to SEQ ID NO: 2 is at least about 99%.

14. An isolated GPR56 polypeptide comprising the amino acid sequence set forth in SEQ ID NO: 2.

15. An antibody that binds to the isolated polypeptide of claim 11.

16. A nucleic acid probe for detecting RNA encoding a GPR56 polypeptide in a sample, said probe comprising at least about 20 contiguous nucleotides of the nucleotide sequence set forth in SEQ ID NO: 1 or a complementary nucleotide sequence thereto.

17. The nucleic acid probe of claim 16 comprising a nucleotide sequence selected from the group consisting of:
(i) a nucleotide sequence comprising nucleotide residues 131-1400 of SEQ ID NO: 1;
(ii) a nucleotide sequence comprising nucleotide residues 1423-2239 of SEQ ID NO: 1;
(iii) a nucleotide sequence comprising nucleotide residues 2264-2282 of SEQ ID NO: 1; and
(iv) a nucleotide sequence comprising about 20-30 contiguous nucleotides of any one of (i) through (iv).

18. A nucleic acid probe comprising a nucleotide sequence selected from the group consisting of:
(i) the sequence set forth in SEQ ID NO: 11;
(ii) the sequence set forth in SEQ ID NO: 12; and
(iii) the sequence set forth in SEQ ID NO: 13.

19. The nucleic acid probe of claim 16 comprising a nucleotide sequence selected from the group consisting of:
(i) a nucleotide sequence that is complementary to nucleotide residues 131-1400 of SEQ ID NO: 1;
(ii) a nucleotide sequence that is complementary to nucleotide residues 1423-2239 of SEQ ID NO: 1;
(iii) a nucleotide sequence that is complementary to nucleotide residues 2264-2282 of SEQ ID NO: 1; and
(iv) a nucleotide sequence comprising at least 20 contiguous nucleotides of any one of (i) through (iv).

20. A nucleic acid probe comprising a nucleotide sequence selected from the group consisting of SEQ ID NO: 14, SEQ ID NO: 15, SEQ ID NO: 16, SEQ ID NO: 17, SEQ ID NO: 18, and SEQ ID NO: 19.

21. A method for detecting a cancer cell in a subject, said method comprising:
(i) determining the level of GPR56 mRNA expressed in a test sample from said subject; and
(ii) comparing the level of GPR56 mRNA determined at (i) to the level of GPR56 mRNA expressed in a comparable sample from a healthy or normal individual,
wherein a level of GPR56 mRNA at (i) that is enhanced in the test sample relative to the comparable sample from the normal or healthy individual is indicative of the presence of a cancer cell in said subject.

22. The method of claim 21 wherein the cancer is a carcinoma.

23. The method of claim 22 wherein the carcinoma is a carcinoma of a tissue selected from the group consisting of: prostate, omentum, ovary, liver, placenta, and brain.

24. The method of claim 21 wherein the samples comprise cells derived from a tissue or selected from the group consisting of: ovary, prostate, kidney, uterus, placenta, cervix, omentum, rectum, brain, bone, lung, lymph, and blood, or urine, semen, abdominal fluid, or serum, or a cell preparation or nucleic acid preparation derived therefrom.

25. A method for detecting ovarian cancer or a metastases thereof in a subject, said method comprising:
(i) determining the level of GPR56 mRNA expressed in a test sample from said subject; and
(ii) comparing the level of GPR56 mRNA determined at (i) to the level of GPR56 mRNA expressed in a comparable sample from a healthy or normal individual,
wherein a level of GPR56 mRNA at (i) that is enhanced in the test sample relative to the comparable sample from the normal or healthy individual is indicative of the presence of ovarian cancer or a metastases thereof in said subject.

26. The method of claim 25 comprising:
(i) hybridizing a GPR56 probe to GPR56-encoding RNA in both the test sample and the comparable sample from the normal or healthy individual under at least low stringency hybridization conditions;
(ii) detecting the hybridization using a detection means; and
(iii) comparing the hybridization signals produced for each sample.

27. The method of claim 25 wherein the sample comprises serum or abdominal fluid, or a tissue selected from the group consisting of: ovary, lymph, lung, liver, brain, placenta, brain, and omentum.

28. The method of claim 25 wherein the sample comprises cells from the ovary or ovarian surface epithelium or cells from the omentum.

29. The method of claim 26 wherein the probe is labeled and the detection means comprises detecting said label following hybridization.

30. The method of claim 29 wherein the GPR56 probe comprises a nucleotide sequence complementary to SEQ ID NO: 1.

31. The method of claim 29 wherein the GPR56 probe comprises a nucleotide sequence selected from the group consisting of: SEQ ID NO: 14, SEQ ID NO: 15, SEQ ID NO: 16, SEQ ID NO: 17, SEQ ID NO: 18, and SEQ ID NO: 19.

32. The method of claim 26 wherein the detection means comprises reverse transcription polymerase chain reaction (RT-PCR).

33. The method of claim 32 wherein one or more sense GPR56 probes comprises a nucleotide sequence selected from the group consisting of SEQ ID NO: 11, SEQ ID NO: 12, and SEQ ID NO: 13.

34. The method of claim 32 wherein one or more antisense GPR56 probes comprises a nucleotide sequence selected from the group consisting of SEQ ID NO: 14, SEQ ID NO: 15, SEQ ID NO: 16, SEQ ID NO: 17, SEQ ID NO: 18, and SEQ ID NO: 19.

35. A method for determining an effector memory T cell response in a subject, said method comprising:
(i) determining the level of GPR56 mRNA expressed in a test sample from said subject; and
(ii) comparing the level of GPR56 mRNA determined at (i) to the level of GPR56 mRNA expressed in a comparable sample from a healthy or normal individual,
wherein a level of GPR56 mRNA at (i) that is enhanced in the test sample relative to the comparable sample from the normal or healthy individual is indicative of an effector memory T cell response in the subject.

36. The method of claim 35 wherein the effector memory T cell response is indicative of inflammatory disease or inflammation in the subject.

37. A method for determining whether or not a subject has been re-infected with an infectious agent, said method comprising:
(i) determining the level of GPR56 mRNA expressed in a test sample from said subject; and
(ii) comparing the level of GPR56 mRNA determined at (i) to the level of GPR56 mRNA expressed in a comparable sample from a healthy or normal individual,
wherein a level of GPR56 mRNA at (i) that is enhanced in the test sample relative to the comparable sample from the normal or healthy individual is indicative of re-infection in the subject.

38. A method for determining the presence of effector memory T cells in a test sample, said method comprising:
(i) hybridizing a GPR56 probe to GPR56-encoding RNA in the test sample under at least low stringency hybridization conditions; and
(ii) detecting the hybridization using a detection means,
wherein said hybridization is indicative of the presence of an effector memory T cell in said test sample.

39. The method of claim 38 wherein the test sample comprises blood or whole serum or a fraction thereof comprising T cells.

40. The method of claim 38 wherein the test sample comprises peripheral blood mononuclear cells (PBMC).

41. A process for counting effector memory T cells in a subject comprising performing the method of claim 38 on a sample from said subject and normalizing the hybridization signal to determine T cell count.

42. A method for detecting a cancer cell in a subject, said method comprising:
(i) determining the level of a GPR56 polypeptide in a test sample from said subject; and
(ii) comparing the level of GPR56 polypeptide determined at (i) to the level of said GPR56 polypeptide in a comparable sample from a healthy or normal individual,
wherein a level of said GPR56 polypeptide at (i) that is enhanced in the test sample relative to the comparable sample from the normal or healthy individual is indicative of the presence of a cancer cell in said subject.

43. The method of claim 42 wherein the level of GPR56 polypeptide is determined by a process comprising:
(i) contacting a sample with an antibody that binds to a GPR56 polypeptide under conditions sufficient for binding to occur; and
(ii) determining the binding.

44. A method for determining whether or not a subject has been re-infected with an infectious agent, said method comprising:
(i) determining the level of a GPR56 polypeptide in a test sample from said subject; and
(ii) comparing the level of the GPR56 polypeptide determined at (i) to the level of said GPR56 polypeptide in a comparable sample from a healthy or normal individual,
wherein a level of said GPR56 polypeptide at (i) that is enhanced in the test sample relative to the comparable sample from the normal or healthy individual is indicative of re-infection in the subject.

45. The method of claim 44 wherein the level of GPR56 polypeptide is determined by a process comprising:
(i) contacting a sample with an antibody that binds to a GPR56 polypeptide under conditions sufficient for binding to occur; and
(ii) determining the binding.

46. A method for determining the presence of effector memory T cells in a test sample, said method comprising:
(i) contacting said sample with an antibody that binds to a GPR56 polypeptide under conditions sufficient for binding to occur; and
(ii) determining the binding.
wherein binding of the antibody to the test sample is indicative of the presence of an effector memory T cell in said test sample.


SEQUENCE LISTING (20 sequences)

SEQ ID NO: 1 - DNA, 2822 bp, Human GPR56-1(TSR32)
  CDS (163)..(2241)
cggcagcagggtctcgctctgtcacacaggctggagtgcagtggtgtgatcttggctcatcgtaacctccacctcccgggttcaagtgattctcatgcctcagcctcccgagtagctgggattacaggtggtgacttccaagagtgactccgtcggaggaaaatgactccccagtcgctgctgcagacgacactgttcctgctgagtctgctcttcctggtccaaggtgcccacggcaggggccacagggaagactttcgcttctgcagccagcggaaccagacacacaggagcagcctccactacaaacccacaccagacctgcgcatctccatcgagaactccgaagaggccctcacagtccatgcccctttccctgcagcccaccctgcttcccgatccttccctgaccccaggggcctctaccacttctgcctctactggaaccgacatgctgggagattacatcttctctatggcaagcgtgacttcttgctgagtgacaaagcctctagcctcctctgcttccagcaccaggaggagagcctggctcagggccccccgctgttagccacttctgtcacctcctggtggagccctcagaacatcagcctgcccagtgccgccagcttcaccttctccttccacagtcctccccacacggccgctcacaatgcctcggtggacatgtgcgagctcaaaagggacctccagctgctcagccagttcctgaagcatccccagaaggcctcaaggaggccctcggctgcccccgccagccagcagttgcagagcctggagtcgaaactgacctctgtgagattcatgggggacatggtgtccttcgaggaggaccggatcaacgccacggtatggaagctccagcccacagccggcctccaggacctgcacatccactcccggcaggaggaggagcagagcgagatcatggagtactcggtgctgctgcctcgaacactcttccagaggacgaaaggccggagcggggaggctgagaagagactcctcctggtggacttcagcagccaagccctgttccaggacaagaattccagccaagtcctgggtgagaaggtcttggggattgtggtacagaacaccaaagtagccaacctcacggagcccgtggtgctcactttccagcaccagctacagccgaagaatgtgactctgcaatgtgtgttctgggttgaagaccccacattgagcagcccggggcattggagcagtgctgggtgtgagaccgtcaggagagaaacccaaacatcctgcttctgcaaccacttgacctactttgcagtgctgatggtctcctcggtggaggtggacgccgtgcacaagcactacctgagcctcctctcctacgtgggctgtgtcgtctctgccctggcctgccttgtcaccattgccgcctacctctgctccagggtgcccctgccgtgcaggaggaaacctcgggactacaccatcaaggtgcacatgaacctgctgctggccgtcttcctgctggacacgagcttcctgctcagcgagccggtggccctgacaggctctgaggctggctgccgagccagtgccatcttcctgcacttctccctgctcacctgcctttcctggatgggcctcgaggggtacaacctctaccgactcgtggtggaggtctttggcacctatgtccctggctacctactcaagctgagcgccatgggctggggcttccccatctttctggtgacgctggtggccctggtggatgtggacaactatggccccatcatcttggctgtgcataggactccagagggcgtcatctacccttccatgtgctggatccgggactccctggtcagctacatcaccaacctgggcctcttcagcctggtgtttctgttcaacatggccatgctagccaccatggtggtgcagatcctgcggctgcgcccccacacccaaaagtggtcacatgtgctgacactgctgggcctcagcctggtccttggcctgccctgggccttgatcttcttctcctttgcttctggcaccttccagcttgtcgtcctctaccttttcagcatcatcacctccttccaaggcttcctcatcttcatctggtactggtccatgcggctgcaggcccggggtggcccctcccctctgaagagcaactcagactgcgccaggctccccatcagctcgggcagcacctcgtccagccgcatctaggcctccagcccacctgcccatgtgatgaagcagagatgcggcctcgtcgcacactgcctgtggcccccgagccaggcccagccccaggccagtcagccgcagactttggaaagcccaacgaccatggagagatgggccgttgccatggtggacggactcccggggctggggcttttgaattggccttggggactactcggctctcactcagctcccacgggactcagaagtgcgccgccatgctgcctagggtactgtccccacatctgtcccaacccagctggaggcctggtctctccttacaacccctgggcccagcctcattgctgggggccaggccttggatcttgagggtctggcacatccttaatcctgtgcccctgcctgggacagaaatgtggctccagttgctctgtctctcgtggtcaccctgagggcactctgcatcctctgtcattttaacctcaggtggcacccagggcgaatggggcccagggcagaccttcagggccagagccctggcggaggagaggccctttgccaggagcacagcagcagctcgcctacctctgagcccg

SEQ ID NO: 2 - protein, 693 aa, Human GPR56-1(TSR32)
MTPQSLLQTTLFLLSLLFLVQGAHGRGHREDFRFCSQRNQTHRSSLHYKPTPDLRISIENSEEALTVHAPFPAAHPASRSFPDPRGLYHFCLYWNRHAGRLHLLYGKRDFLLSDKASSLLCFQHQEESLAQGPPLLATSVTSWWSPQNISLPSAASFTFSFHSPPHTAAHNASVDMCELKRDLQLLSQFLKHPQKASRRPSAAPASQQLQSLESKLTSVRFMGDMVSFEEDRINATVWKLQPTAGLQDLHIHSRQEEEQSEIMEYSVLLPRTLFQRTKGRSGEAEKRLLLVDFSSQALFQDKNSSQVLGEKVLGIVVQNTKVANLTEPVVLTFQHQLQPKNVTLQCVFWVEDPTLSSPGHWSSAGCETVRRETQTSCFCNHLTYFAVLMVSSVEVDAVHKHYLSLLSYVGCVVSALACLVTIAAYLCSRVPLPCRRKPRDYTIKVHMNLLLAVFLLDTSFLLSEPVALTGSEAGCRASAIFLHFSLLTCLSWMGLEGYNLYRLVVEVFGTYVPGYLLKLSAMGWGFPIFLVTLVALVDVDNYGPIILAVHRTPEGVIYPSMCWIRDSLVSYITNLGLFSLVFLFNMAMLATMVVQILRLRPHTQKWSHVLTLLGLSLVLGLPWALIFFSFASGTFQLVVLYLFSIITSFQGFLIFIWYWSMRLQARGGPSPLKSNSDCARLPISSGSTSSSRI

SEQ ID NO: 3 - DNA, 3711 bp, human GPR56-2
  CDS (168)..(2246)
ggcacgaggtggagggtctcgctctgtcacacaggctggagtgcagtggtgtgatcttggctcatcgtaacctccacctcccgggttcaagtgattctcatgcctcagcctcccgagtagctgggattacaggtggtgacttccaagagtgactccgtcggaggaaaatgactccccagtcgctgctgcagacgacactgttcctgctgagtctgctcttcctggtccaaggtgcccacggcaggggccacagggaagactttcgcttctgcagccagcggaaccagacacacaggagcagcctccactacaaacccacaccagacctgcgcatctccatcgagaactccgaagaggccctcacagtccatgcccctttccctgcagcccaccctgcttcccgatccttccctgaccccaggggcctctaccacttctgcctctactggaaccgacatgctgggagattacatcttctctatggcaagcgtgacttcttgctgagtgacaaagcctctagcctcctctgcttccagcaccaggaggagagcctggctcagggccccccgctgttagccacttctgtcacctcctggtggagccctcagaacatcagcctgcccagtgccgccagcttcaccttctccttccacagtcctccccacacggccgctcacaatgcctcggtggacatgtgcgagctcaaaagggacctccagctgctcagccagttcctgaagcatccccagaaggcctcaaggaggccctcggctgcccccgccagccagcagttgcagagcctggagtcgaaactgacctctgtgagattcatgggggacatggtgtccttcgaggaggaccggatcaacgccacggtgtggaagctccagcccacagccggcctccaggacctgcacatccactcccggcaggaggaggagcagagcgagatcatggagtactcggtgctgctgcctcgaacactcttccagaggacgaaaggccggagcggggaggctgagaagagactcctcctggtggacttcagcagccaagccctgttccaggacaagaattccagccacgtcctgggtgagaaggtcttggggattgtggtacagaacaccaaagtagccaacctcacggagcccgtggtgctcaccttccagcaccagctacagccgaagaatgtgactctgcaatgtgtgttctgggttgaagaccccacattgagcagcccggggcattggagcagtgctgggtgtgagaccgtcaggagagaaacccaaacatcctgcttctgcaaccacttgacctactttgcagtgctgatggtctcctcggtggaggtggacgccgtgcacaagcactacctgagcctcctctcctacgtgggctgtgtcgtctctgccctggcctgccttgtcaccattgccgcctacctctgctccagggtgcccctgccgtgcaggaggaaacctcgggactacaccatcaaggtgcacatgaacctgctgctggccgtcttcctgctggacacgagcttcctgctcagcgagccggtggccctgacaggctctgaggctggctgccgagccagtgccatcttcctgcacttctccctgctcacctgcctttcctggatgggcctcgaggggtacaacctctaccgactcgtggtggaggtctttggcacctatgtccctggctacctactcaagctgagcgccatgggctggggcttccccatctttctggtgacgctggtggccctggtggatgtggacaactatggccccatcatcttggctgtgcataggactccagagggcgtcatctacccttccatgtgctggatccgggactccctggtcagctacatcaccaacctgggcctcttcagcctggtgtttctgttcaacatggccatgctagccaccatggtggtgcagatcctgcggctgcgcccccacacccaaaagtggtcacatgtgctgacactgctgggcctcagcctggtccttggcctgccctgggccttgatcttcttctcctttgcttctggcaccttccagcttgtcgtcctctaccttttcagcatcatcacctccttccaaggcttcctcatcttcatctggtactggtccatgcggctgcaggcccggggtggcccctcccctctgaagagcaactcagacagcgccaggctccccatcagctcgggcagcacctcgtccagccgcatctaggcctccagcccacctgcccatgtgatgaagcagagatgcggcctcgtcgcacactgcctgtggcccccgagccaggcccagccccaggccagtcagccgcagactttggaaagcccaacgaccatggagagatgggccgttgccatggtggacggactcccgggctgggcttttgaattggccttggggactactcggctctcactcagctcccacgggactcagaagtgcgccgccatgctgcctagggtactgtccccacatctgtcccaacccagctggaggcctggtctctccttacaaaccctgggcccagccctcattgctgggggccaggccttggatcttgagggtctggcacatccttaatcctgtgcccctgcctgggacagaaatgtggctccagttgctctgtctctcgtggtcaccctgagggcactctgcatcctctgtcattttaacctcaggtggcacccagggcgaatggggcccagggcagaccttcagggccagagccctggcggaggagaggccctttgccaggagcacagcagcagctcgcctacctctgagcccaggccccctccctccctcagccccccagtcctccctccatcttccctggggttctcctcctctcccagggcctccttgctccttcgttcacagctgggggtccccgattccaatgctgttttttggggagtggtttccaggagctgcctggtgtctgctgtaaatgtttgtctactgcacaagcctcggcctgcccctgagccaggctcggtaccgatgcgtgggctgggctaggtccctctgtccatctgggcctttgtatgagctgcattgcccttgctcaccctgaccaagcacacgcctcagaggggccctcagcctctcctgaagccctcttgtggcaagaactgtggaccatgccagtcccgtctggtttccatcccaccactccaaggactgagactgacctcctctggtgacactggcctagagcctgacactctcctaagaggttctctccaagcccccaaatagctccaggcgccctcggccgcccatcatggttaattctgtccaacaaacacacacgggtagattgctggcctgttgtaggtggtagggacacagatgaccgacctggtcactcctcctgccaacattcagtctggtatgtgaggcgtgcgtgaagcaagaactcctggagctacagggacagggagccatcattcctgcctgggaatcctggaagacttcctgcaggagtcagcgttcaatcttgaccttgaagatgggaaggatgttctttttacgtaccaattcttttgtcttttgatattaaaaagaagtacatgttcattgtagagaatttggaaactgtagaagagaatcaagaagaaaaataaaaatcagctgttgtaatcgcctagcaaaaaaaaaaaaaaaaaaa

SEQ ID NO: 4 - protein, 693 aa, human GPR56-2
MTPQSLLQTTLFLLSLLFLVQGAHGRGHREDFRFCSQRNQTHRSSLHYKPTPDLRISIENSEEALTVHAPFPAAHPASRSFPDPRGLYHFCLYWNRHAGRLHLLYGKRDFLLSDKASSLLCFQHQEESLAQGPPLLATSVTSWWSPQNISLPSAASFTFSFHSPPHTAAHNASVDMCELKRDLQLLSQFLKHPQKASRRPSAAPASQQLQSLESKLTSVRFMGDMVSFEEDRINATVWKLQPTAGLQDLHIHSRQEEEQSEIMEYSVLLPRTLFQRTKGRSGEAEKRLLLVDFSSQALFQDKNSSHVLGEKVLGIVVQNTKVANLTEPVVLTFQHQLQPKNVTLQCVFWVEDPTLSSPGHWSSAGCETVRRETQTSCFCNHLTYFAVLMVSSVEVDAVHKHYLSLLSYVGCVVSALACLVTIAAYLCSRVPLPCRRKPRDYTIKVHMNLLLAVFLLDTSFLLSEPVALTGSEAGCRASAIFLHFSLLTCLSWMGLEGYNLYRLVVEVFGTYVPGYLLKLSAMGWGFPIFLVTLVALVDVDNYGPIILAVHRTPEGVIYPSMCWIRDSLVSYITNLGLFSLVFLFNMAMLATMVVQILRLRPHTQKWSHVLTLLGLSLVLGLPWALIFFSFASGTFQLVVLYLFSIITSFQGFLIFIWYWSMRLQARGGPSPLKSNSDSARLPISSGSTSSSRI

SEQ ID NO: 5 - DNA, 2816 bp, human GPR56-3
  CDS (159)..(2237)
agcagggtctcactctgtcacccaggctggagtgcagtggcgcagttatagctcactgcagccttgaactcctgggctcaagtgatccttccacctcagcctccctagtagctgggaccacaggtggtgacttccaagagtgactccgtcggaggaaaatgactccccagtcgctgctgcagacgacactgttcctgctgagtctgctcttcctggtccaaggtgcccacggcaggggccacagggaagactttcgcttctgcagccagcggaaccagacacacaggagcagcctccactacaaacccacaccagacctgcgcatctccatcgagaactccgaagaggccctcacagtccatgcccctttccctgcagcccaccctgcttcccgatccttccctgaccccaggggcctctaccacttctgcctctactggaaccgacatgctgggagattacatcttctctatggcaagcgtgacttcttgctgagtgacaaagcctctagcctcctctgcttccagcaccaggaggagagcctggctcagggccccccgctgttagccacttctgtcacctcctggtggagccctcagaacatcagcctgcccagtgccgccagcttcaccttctccttccacagtcctccccacacggccgctcacaatgcctcggtggacatgtgcgagctcaaaagggacctccagctgctcagccagttcctgaagcatccccagaaggcctcaaggaggccctcggctgcccccgccagccagcagttgcagagcctggagtcgaaactgacctctgtgagattcatgggggacatggtgtccttcgaggaggaccggatcaacgccacggtgtggaagctccagcccacagccggcctccaggacctgcacatccactcccggcaggaggaggagcagagcgagatcatggagtactcggtgctgctgcctcgaacactcttccagaggacgaaaggccggagcggggaggctgagaagagactcctcctggtggacttcagcagccaagccctgttccaggacaagaattccagccaagtcctgggtgagaaggtcttggggattgtggtacagaacaccaaagtagccaacctcacggagcccgtggtgctcactttccagcaccagctacagccgaagaatgtgactctgcaatgtgtgttctgggttgaagaccccacattgagcagcccggggcattggagcagtgctgggtgtgagaccgtcaggagagaaacccaaacatcctgcttctgcaaccacttgacctactttgcagtgctgatggtctcctcggtggaggtggacgccgtgcacaagcactacctgagcctcctctcctacgtgggctgtgtcgtctctgccctggcctgccttgtcaccattgccgcctacctctgctccagggtgcccctgccgtgcaggaggaaacctcgggactacaccatcaaggtgcacatgaacctgctgctggccgtcttcctgctggacacgagcttcctgctcagcgagccggtggccctgacaggctctgaggctggctgccgagccagtgccatcttcctgcacttctccctgctcacctgcctttcctggatgggcctcgaggggtacaacctctaccgactcgtggtggaggtctttggcacctatgtccctggctacctactcaagctgagcgccatgggctggggcttccccatctttctggtgacgctggtggccctggtggatgtggacaactatggccccatcatcttggctgtgcataggactccagagggcgtcatctacccttccatgtgctggatccgggactccctggtcagctacatcaccaacctgggcctcttcagcctggtgtttctgttcaacatggccatgctagccaccatggtggtgcagatcctgcggctgcgcccccacacccaaaagtggtcacatgtgctgacactgctgggcctcagcctggtccttggcctgccctgggccttgatcttcttctcctttgcttctggcaccttccagcttgtcgtcctctaccttttcagcatcatcacctccttccaaggcttcctcatcttcatctggtactggtccatgcggctgcaggcccggggtggcccctcccctctgaagagcaactcagacagcgccaggctccccatcagctcgggcagcacctcgtccagccgcatctaggcctccagcccacctgcccatgtgatgaagcagagattcggcctcgtcgcacactgcctgtggcccccgagcccggcccagccccaggccagtcagccgcagactttggaaagcccaacgaccatggagagatgggccgttgccatggtggacggactcccgggctgggcttttgaattggccttggggactactcggctctcactcagctcccacgggactcagaagtgcgccgccatgctgcctagggtactgtccccacatctgtcccaacccagctggaggcctggtctctccttacaacccctgggcccagccctcattgctgggggccaggccttggatcttgagggtctggcacatccttaatcctgtgcccctgcctgggacagaaatgtggctccagttgctctgtctctcgtggtcaccctgagggcactctgcatcctctgtcattttaacctcaggtggcacccagggcgaatggggcccagggcagaccttcagggccagagccctggcggaggagaggccctttgccaggagcacagcagcagctcgcctacctctgagccc

SEQ ID NO: 6 - protein, 693 aa, human GPR56-3
MTPQSLLQTTLFLLSLLFLVQGAHGRGHREDFRFCSQRNQTHRSSLHYKPTPDLRISIENSEEALTVHAPFPAAHPASRSFPDPRGLYHFCLYWNRHAGRLHLLYGKRDFLLSDKASSLLCFQHQEESLAQGPPLLATSVTSWWSPQNISLPSAASFTFSFHSPPHTAAHNASVDMCELKRDLQLLSQFLKHPQKASRRPSAAPASQQLQSLESKLTSVRFMGDMVSFEEDRINATVWKLQPTAGLQDLHIHSRQEEEQSEIMEYSVLLPRTLFQRTKGRSGEAEKRLLLVDFSSQALFQDKNSSQVLGEKVLGIVVQNTKVANLTEPVVLTFQHQLQPKNVTLQCVFWVEDPTLSSPGHWSSAGCETVRRETQTSCFCNHLTYFAVLMVSSVEVDAVHKHYLSLLSYVGCVVSALACLVTIAAYLCSRVPLPCRRKPRDYTIKVHMNLLLAVFLLDTSFLLSEPVALTGSEAGCRASAIFLHFSLLTCLSWMGLEGYNLYRLVVEVFGTYVPGYLLKLSAMGWGFPIFLVTLVALVDVDNYGPIILAVHRTPEGVIYPSMCWIRDSLVSYITNLGLFSLVFLFNMAMLATMVVQILRLRPHTQKWSHVLTLLGLSLVLGLPWALIFFSFASGTFQLVVLYLFSIITSFQGFLIFIWYWSMRLQARGGPSPLKSNSDSARLPISSGSTSSSRI

SEQ ID NO: 7 - DNA, 3835 bp, human GPR56-4
  CDS (307)..(2367)
ccagcttcaaagtctctgtcctcttgaaaaaaggtggtcgggggacattgacccaccagccccgcaggctactgcctgcaaacaagaacccctcatctgccacgcacgttcttaatgtatctatagtctgcctgatgccacaaaggagtccagggtctcgctctgtcacacaggctggagtgcagtggtgtgatcttggctcatcgtaacctccacctcccgggttcaagtgattctcatgcctcagcctcccgagtagctgggattacaggtggtgacttccaagagtgactccgtcggaggaaaatgactccccagtcgctgctgcagacgacactgttcctgctgagtctgctcttcctggtccaaggtgcccacggcaggggccacagggaagactttcgcttctgcagccagcggaaccagacacacaggagcagcctccactacaaacccacaccagacctgcgcatctccatcgagaactccgaagaggccctcacagtccatgcccctttccctgcagcccaccctgcttcccgatccttccctgaccccaggggcctctaccacttctgcctctactggaaccgacatgctgggagattacatcttctctatggcaagcgtgacttcttgctgagtgacaaagcctctagcctcctctgcttccagcaccaggaggagagcctggctcagggccccccgctgttagccacttctgtcacctcctggtggagccctcagaacatcagcctgcccagtgccgccagcttcaccttctccttccacagtcctccccacacggccgctcacaatgcctcggtggacatgtgcgagctcaaaagggacctccagctgctcagccagttcctgaagcatccccagaaggcctcaaggaggccctcggctgcccccgccagccagcagttgcagagcctggagtcgaaactgacctctgtgagattcatgggggacatggtgtccttcgaggaggaccggatcaacgccacggtgtggaagctccagcccacagccggcctccaggacctgcacatccactcccggcaggaggaggagcagagcgagatcatggagtactcggtgctgctgcctcgaacactcttccagaggacgaaaggccggagcggggaggctgagaagagactcctcctggtggacttcagcagccaagccctgttccaggacaagaattccagccaagtcctgggtgagaaggtcttggggattgtggtacagaacaccaaagtagccaacctcacggagcccgtggtgctcactttccagcaccagctacagccgaagaatgtgactctgcaatgtgtgttctgggttgaagaccccacattgagcagcccggggcattggagcagtgctgggtgtgagaccgtcaggagagaaacccaaacatcctgcttctgcaaccacttgacctactttgcagtgctgatggtctcctcggtggaggtggacgccgtgcacaagcactacctgagcctcctctcctacgtgggctgtgtcgtctctgccctggcctgccttgtcaccattgccgcctacctctgctccaggaggaaacctcgggactacaccatcaaggtgcacatgaacctgctgctggccgtcttcctgctggacacgagcttcctgctcagcgagccggtggccctgacaggctctgaggctggctgccgagccagtgccatcttcctgcacttctccctgctcacctgcctttcctggatgggcctcgaggggtacaacctctaccgactcgtggtggaggtctttggcacctatgtccctggctacctactcaagctgagcgccatgggctggggcttccccatctttctggtgacgctggtggccctggtggatgtggacaactatggccccatcatcttggctgtgcataggactccagagggcgtcatctacccttccatgtgctggatccgggactccctggtcagctacatcaccaacctgggcctcttcagcctggtgtttctgttcaacatggccatgctagccaccatggtggtgcagatcctgcggctgcgcccccacacccaaaagtggtcacatgtgctgacactgctgggcctcagcctggtccttggcctgccctgggccttgatcttcttctcctttgcttctggcaccttccagcttgtcgtcctctaccttttcagcatcatcacctccttccaaggcttcctcatcttcatctggtactggtccatgcggctgcaggcccggggtggcccctcccctctgaagagcaactcagacagcgccaggctccccatcagctcgggcagcacctcgtccagccgcatctaggcctccagcccacctgcccatgtgatgaagcagagatgcggcctcgtcgcacactgcctgtggcccccgagccaggcccagccccaggccagtcagccgcagactttggaaagcccaacgaccatggagagatgggccgttgccatggtggacggactcccgggctgggcttttgaattggccttggggactactcggctctcactcagctcccacgggactcagaagtgcgccgccatgctgcctagggtactgtccccacatctgtcccaacccagctggaggcctggtctctccttacaacccctgggcccagccctcattgctgggggccaggccttggatcttgagggtctggcacatccttaatcctgtgcccctgcctgggacagaaatgtggctccagttgctctgtctctcgtggtcaccctgagggcactctgcatcctctgtcattttaacctcaggtggcacccagggcgaatggggcccagggcagaccttcagggccagagccctggcggaggagaggccctttgccaggagcacagcagcagctcgcctacctctgagcccaggccccctccctccctcagccccccagtcctccctccatcttccctggggttctcctcctctcccagggcctccttgctccttcgttcacagctgggggtccccgattccaatgctgttttttggggagtggtttccaggagctgcctggtgtctgctgtaaatgtttgtctactgcacaagcctcggcctgcccctgagccaggctcggtaccgatgcgtgggctgggctaggtccctctgtccatctgggcctttgtatgagctgcattgcccttgctcaccctgaccaagcacacgcctcagaggggccctcagcctctcctgaagccctcttgtggcaagaactgtggaccatgccagtcccgtctggtttccatcccaccactccaaggactgagactgacctcctctggtgacactggcctagagcctgacactctcctaagaggttctctccaagcccccaaatagctccaggcgccctcggccgcccatcatggttaattctgtccaacaaacacacacgggtagattgctggcctgttgtaggtggtagggacacagatgaccgacctggtcactcctcctgccaacattcagtctggtatgtgaggcgtgcgtgaagcaagaactcctggagctacagggacagggagccatcattcctgcctgggaatcctggaagacttcctgcaggagtcagcgttcaatcttgaccttgaagatgggaaggatgttctttttacgtaccaattcttttgtcttttgatattaaaaagaagtacatgttcattgtagagaatttggaaactgtagaagagaatcaagaagaaaaataaaaatcagctgttgtaatcgcctagcaaactggaaaaaaaaaaaaaaa

SEQ ID NO: 8 - protein, 687 aa, human GPR56-4
MTPQSLLQTTLFLLSLLFLVQGAHGRGHREDFRFCSQRNQTHRSSLHYKPTPDLRISIENSEEALTVHAPFPAAHPASRSFPDPRGLYHFCLYWNRHAGRLHLLYGKRDFLLSDKASSLLCFQHQEESLAQGPPLLATSVTSWWSPQNISLPSAASFTFSFHSPPHTAAHNASVDMCELKRDLQLLSQFLKHPQKASRRPSAAPASQQLQSLESKLTSVRFMGDMVSFEEDRINATVWKLQPTAGLQDLHIHSRQEEEQSEIMEYSVLLPRTLFQRTKGRSGEAEKRLLLVDFSSQALFQDKNSSQVLGEKVLGIVVQNTKVANLTEPVVLTFQHQLQPKNVTLQCVFWVEDPTLSSPGHWSSAGCETVRRETQTSCFCNHLTYFAVLMVSSVEVDAVHKHYLSLLSYVGCVVSALACLVTIAAYLCSRRKPRDYTIKVHMNLLLAVFLLDTSFLLSEPVALTGSEAGCRASAIFLHFSLLTCLSWMGLEGYNLYRLVVEVFGTYVPGYLLKLSAMGWGFPIFLVTLVALVDVDNYGPIILAVHRTPEGVIYPSMCWIRDSLVSYITNLGLFSLVFLFNMAMLATMVVQILRLRPHTQKWSHVLTLLGLSLVLGLPWALIFFSFASGTFQLVVLYLFSIITSFQGFLIFIWYWSMRLQARGGPSPLKSNSDSARLPISSGSTSSSRI

SEQ ID NO: 9 - DNA, 3021 bp, murine GPR56
  CDS (321)..(2384)
agacagcgtgatcccggcctcccacggggcagcttttactgtctagggaagaaatccccaaagtccatggagtctgaagactctgtcaagcctcgctaggaaacctaggagttttagagggcacttggcaccggaagctagccgggtaggcggagcctcacctggattgagttcacagctgcctagacaggctcagactaggtgctgggcacctgggaggaggaggagacattagcagcaaaggctgttaacagaagtgcctgcctaggcttggaggcaagacgctgctgttcacagtgcgagacggaggtaggagtataatggctgtccaggtgctgcggcagatggtctacttcctactgagtctgttttctctggtgcaaggtgcacacagtggcagcccccgagaagacttccgcttctgtggccagcggaaccagacccaacagagcaccctccactatgatcaatcttcagagcctcacatctttgtgtggaacacagaggagaccctcacaattcgtgcccccttcctggcagccccagatattccccgcttcttcccagagcctagagggctctatcacttctgcctctactggagtcgccacactgggagactccacttgcgctatggcaagcatgactacctgcttagtagccaagcctccagactcctctgcttccagaaacaggagcagagcctgaagcagggagccccgctgatcgccacctctgtcagctcctggcagattccccagaacaccagcctgcctggggctccgagcttcatcttctccttccacaatgccccacacaaggtctcccacaatgcatctgtggacatgtgtgatctcaagaaggaattgcagcagcttagcaggtacctgcagcaccctcaaaaggctgccaagcggcccaccgcagcgttcatcagccagcagttacagagcctggagtcaaagctgacctctgtgagcttcctgggagacacattatcctttgaggaggaccgggtcaatgctacagtgtggaagctgccacccacagccggtctagaggatctgcatatccactcccagaaggaggaggagcagagtgaggtccaggcatactcgctgttgcttccccgggccgtattccagcagaccagaggccgtcgccgggatgacgccaagaggctcctggtagtagacttcagcagccaagctttgttccaggacaagaattctagccaagtcctgggtgagaaggtcttgggtattgtcgtgcagaacaccaaagtcaccaacctctcagatccggtggtactcaccttccagcaccagcctcagccaaaaaatgtgactctgcagtgcgtgttctgggttgaagacccggcatcaagcagcacagggagctggagcagtgcaggctgcgagacagtgagcagagacacacagacatcctgcctgtgcaaccacctgacctactttgcagtgctgatggtgtcatccacagaggtagaagccactcacaaacactacctcacgctcctgtcctacgtgggctgtgtcatctctgctctggcttgtgtcttcactatcgctgcctacctctgctccaggaggaagtcacgtgactacaccatcaaagtccacatgaacctgctgtccgctgtcttcctgctggacgtgagcttcctgctcagcgagcctgtggcactgacgggctccgaagcagcctgtcgcaccagtgccatgttcctgcacttctccctgcttgcctgcctctcctggatgggcctcgagggctacaatctctaccgactggtggtggaggtcttcggtacctatgtgcccggctatctgctcaagctgagcatcgtgggctggggttttcctgtcttcctggtcactctggtggcgttggtggatgtgaataactacggccccattatcctagctgtgcgccggactccggaacgtgtcacctacccctctatgtgctggatccgggactccctggtgagctatgtcaccaacctgggcctcttcagtctggtgttcctgttcaacctggctatgctggccaccatggtggtgcagatcctgcggcttcgcccgcacagccagaactggccccacgtgctgaccctgctgggcctcagcctggtccttggcctcccctgggccttggtcttcttttcctttgcttccggcaccttccagcttgtcatcctctacctcttcagcatcataacttcctaccaaggcttcctcatcttcctgtggtactggtccatgcggttccaggcccaaggcggcccctcccctctgaagaacaactcagacagcgccaaactccccatcagctccggcagcacctcctccagccgcatctaagccaccgccacacctcccctccgggaggacacatgcatggcgtccgctcacgatgtctgtggcccagtgctgtgcccacccagcctttgttggttagtggcatactagagaaggccctggtccttgaaggcgtagggctgttgctctgagggacctccatctctcctgaagcctcctccctctggcaagtactgggatacagccaccctttcaacccagcactctgaagaccaagacagccccctctggtgacactggccaagcttgatctttttcctaagaagtggtcttcagatccccgcaggtcgctcagaagacactgggctgcctagtgtgaattctgtcctactaacgtacagtgagcagctcctcacccccacccccgcaaaagctctcaccaagtcctggagtgtcaggcagggggctggaaatccaggaggacttcctgcaaaaggcagcatttcatcttgacctcagccttcaggttggggagaatgttctttttaaataccagttcatttgtcttttgatattaaagctctttatagagagtctggaaactgtaggcgattgtcgagaagagaaataaaaatgagctgttatctaatgccatggcaaagcagcacaaaaaaaaaaa

SEQ ID NO: 10 - protein, 687 aa, murine GPR56
MAVQVLRQMVYFLLSLFSLVQGAHSGSPREDFRFCGQRNQTQQSTLHYDQSSEPHIFVWNTEETLTIRAPFLAAPDIPRFFPEPRGLYHFCLYWSRHTGRLHLRYGKHDYLLSSQASRLLCFQKQEQSLKQGAPLIATSVSSWQIPQNTSLPGAPSFIFSFHNAPHKVSHNASVDMCDLKKELQQLSRYLQHPQKAAKRPTAAFISQQLQSLESKLTSVSFLGDTLSFEEDRVNATVWKLPPTAGLEDLHIHSQKEEEQSEVQAYSLLLPRAVFQQTRGRRRDDAKRLLVVDFSSQALFQDKNSSQVLGEKVLGIVVQNTKVTNLSDPVVLTFQHQPQPKNVTLQCVFWVEDPASSSTGSWSSAGCETVSRDTQTSCLCNHLTYFAVLMVSSTEVEATHKHYLTLLSYVGCVISALACVFTIAAYLCSRRKSRDYTIKVHMNLLSAVFLLDVSFLLSEPVALTGSEAACRTSAMFLHFSLLACLSWMGLEGYNLYRLVVEVFGTYVPGYLLKLSIVGWGFPVFLVTLVALVDVNNYGPIILAVRRTPERVTYPSMCWIRDSLVSYVTNLGLFSLVFLFNLAMLATMVVQILRLRPHSQNWPHVLTLLGLSLVLGLPWALVFFSFASGTFQLVILYLFSIITSYQGFLIFLWYWSMRFQAQGGPSPLKNNSDSAKLPISSGSTSSSRI

SEQ ID NO: 11 - DNA, 22 bp, probe
catctccatcgagaactccgaa

SEQ ID NO: 12 - DNA, 21 bp, probe
ctacgtgggctgtgtcgtctc

SEQ ID NO: 13 - DNA, 23 bp, probe
agcgagatcatggagtactcggt

SEQ ID NO: 14 - DNA, 22 bp, probe
ttcggagttctcgatggagatc

SEQ ID NO: 15 - DNA, 21 bp, probe
gagacgacacagcccacgtag

SEQ ID NO: 16 - DNA, 23 bp, probe
accgagtactccatgatctcgct

SEQ ID NO: 17 - DNA, 21 bp, probe
acctccaccacgagtcggtag

SEQ ID NO: 18 - DNA, 21 bp, probe
aaagacctccaccacgagtcg

SEQ ID NO: 19 - DNA, 25 bp, probe
aaagacctccaccacgagtcggtag

SEQ ID NO: 20 - protein, 10 aa, N-terminal peptide
MTPQSLLQTT